SEQUENCE LISTING (4 sequences)

SEQ ID NO: 1 - DNA, 22 bp, Artificial sequence
  Synthetic peptide
gggctggcccgatgtatttgat

SEQ ID NO: 2 - DNA, 23 bp, Artificial sequence
  Synthetic peptide
atagaggttgtcgaaggctgggc

SEQ ID NO: 3 - DNA, 12 bp, Artificial sequence
  Synthetic peptide
gaggaagaggaa

SEQ ID NO: 4 - DNA, 12 bp, Artificial sequence
  Synthetic peptide
gagctcgagctc

(19) United States
(12) Patent Application Publication
(10) Pub. No.: US 2007/0207975 A1
(43) Pub. Date: Sep. 6, 2007

(54) Materials and methods for treating and preventing Her-2/neu overexpressing, FAS-elevated cancer cells

(75) Inventors: Javier A. Menendez, Evanston, IL (US); Ruth Lupu, Highland Park, IL (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: EVANSTON NORTHWESTERN HEALTHCARE, Evanston, IL (US)

(21) Appl. No.: 11/651,803

(22) Filed: Jan. 9, 2007

Related U.S. Application Data
(60) Provisional application No. 60/757,926, filed on Jan. 10, 2006.

(51) Int. Cl. A61K 48/00; A61K 31/202

(52) U.S. Cl. 514/44; 514/560

(57) ABSTRACT

The invention provides methods for treating cancer cells overexpressing Her-2/neu, the product of the erbB-2 gene, comprising administering a therapeutically effective amount of an unsaturated fatty acid. The cancer cells amenable to treatment exhibit relatively elevated activity levels of fatty acid synthase. Exemplary fatty acids useful in the methods of the invention include ω-9 monounsaturated fatty acids (e.g., oleic acid), ω-6 polyunsaturated fatty acids (e.g., γ-linolenic acid) and ω-3 polyunsaturated fatty acids (e.g., α-linolenic acid). The invention also provides methods of preventing cancer comprising administering a prophylactically effective amount of a fatty acid and kits for preventing and/or treating cancer comprising a fatty acid and a conventional anti-cancer therapeutic.

The present application claims the benefit of priority of U.S. Provisional Application No. 60/757,926, which was filed Jan. 10, 2006 and is specifically incorporated herein by reference in its entirety.

This invention was made with U.S. government support and the U.S. government may have certain rights in the invention pursuant to the terms of Grant No. BC033538 from the Dept. of Defense and Grant No. P50CA89018-03 from the National Institutes of Health.

### FIELD

The invention relates to the field of medicine and, more particularly, to the field of cancer medicine.

### BACKGROUND

Epidemiological studies indicate that women in countries with high-fat diets have a risk of breast cancer that can be five-fold higher than that of women in countries with low-fat consumption [1-3], strongly suggesting that a high intake of dietary fat could increase breast cancer risk [3]. This dietary fat hypothesis has been supported by a number of epidemiological, experimental and mechanistic data, collectively providing evidence that dietary or exogenously provided fats may play a role in the carcinogenesis, evolution and/or progression of breast cancer [3-5]. However, case-control, cohort and recent prospective epidemiological studies have generated conflicting results, and taken together do not support a strong association [6-9].

Research in experimental animals has yielded inconsistent results, having attributed a range of effects of dietary fat extending from a non-promoting or a low-promoting effect to a protective one on breast cancer [1, 14-16]. These conflicting results may be explained in part by the fact that olive oil is administered as a mixture of oil containing several fatty acids and glycerol, as well as natural chemoprotectants (tocopherols, carotenoids, polyphenols, and the like) [17-19], e.g., antioxidants in the unsaponifiable fraction of the oil.

The Her-2/neu oncogene (also called neu and erbB-2) represents one of the most important oncogenes in breast cancer. Her-2/neu codes for the p185^{Her-2/neu} oncoprotein, a transmembrane tyrosine kinase orphan receptor [22, 23]. Her-2/neu amplification and overexpression occurs in 20% of breast carcinomas and is correlated with unfavorable clinical outcome [24-26]. Expression of high levels of Her-2/neu is sufficient to induce the neoplastic transformation of some cell lines [27, 28], suggesting a role for Her-2/neu in the etiology of some breast carcinomas. Indeed, Her-2/neu is overexpressesed not only in invasive breast cancer, but also in pre-neoplastic breast lesions, such as atypical duct proliferations and in ductal carcinoma of the breast in situ [29-31]. Moreover, Her-2/neu is a metastasis-promoting gene, enhancing the invasive and metastatic phenotype of breast cancer cells [32, 33]. Her-2/neu overexpression is also associated with resistance to chemo- and endocrine therapies [34, 35], while representing a successful therapeutic target of the biotechnology era, as exemplified by the drug trastuzumab (Herceptin®; Genentech, San Francisco, Calif.). Trastuzumab is a humanized monoclonal IgG1, binding with high affinity to the ectodomain of p185^{Her-2/neu} that has clinical activity in a subset of breast cancer patients, thus confirming the role of Her-2/neu in the progression of some breast carcinomas [36-39].

Although data suggest that trastuzumab may be useful in select cases of advanced breast cancer, these benefits are modest and usually do not represent a cure. Moreover, not all Her-2/neu-overexpressing respond to treatment with trastuzumab and its clinical benefit is limited by the fact that resistance develops rapidly in virtually all treated patients [40]. Although the molecular mechanisms underlying trastuzumab resistance have begun to emerge [41-43], there are no data concerning strategies able to sensitize breast cancer cells to the growth-inhibitory activity of anti-p185^{Her-2/neu} antibodies, such as trastuzumab.

Thus, a need continues to exist in the art for effective, and preferably safe, approaches to the treatment of a variety of cancers, including breast carcinomas, in man and other animals. This need is so pronounced that single-therapeutic, as well as combination therapies, are desperately being sought.

### SUMMARY

The invention provides materials and methods useful in treating a variety of cancers, as well as preventing such cancers and ameliorating at least one symptom associated with such cancers, by administering a therapeutically or prophylactically effective amount of an unsaturated C16-C22 trans-fatty acid. The cancers amenable to the methods of the invention overexpress both p185^{Her-2/neu}, the erbB-2 (neu) gene product, and fatty acid synthase relative to non-cancerous cells of the same type. The materials and methods of the invention are suitable for use in combination with known anti-cancer therapeutics and therapies, particularly those that reduce the activity of p185^{Her-2/neu}. An exemplary known anti-cancer therapeutic contemplated for combination with the materials and/or methods of the invention is an antibody specifically recognizing p185^{Her-2/neu}, such as trastuzumab.

In one aspect, the invention provides a method of treating a cancer cell overexpressing p185^{Her-2/neu} and fatty acid synthase comprising administering a therapeutically effective amount of an unsaturated trans-fatty acid to an organism comprising the cancer cell. In some embodiments, a nucleic acid overexpressing p185^{Her-2/neu} comprises a promoter comprising a PEA3 binding site. This aspect of the invention comprehends methods wherein the organism in need is a human. An exemplary cancer amenable to treatment by the method include a cancer selected from the group consisting of breast cancer, ovarian cancer, prostate cancer, colorectal cancer, bladder cancer, stomach cancer, lung cancer, oral cancer of the tongue and cancer of the endometrium. Consistent with the statement above, the method embraces embodiments wherein the fatty acid is selected from the group consisting of C16-C22 fatty acids. Preferably, the fatty acid is a naturally occurring mono- or polyunsaturated trans-fatty acid. In particular, embodiments of the method are contemplated wherein the fatty acid is selected from the group consisting of an omega-9 unsaturated fatty acid, an omega-6 unsaturated fatty acid and an omega-3 unsaturated fatty acid. Exemplary fatty acids suitable for use in the method include a fatty acid selected from the group consisting of oleic acid, γ-linolenic acid and α-linolenic acid.

Another aspect of the invention is drawn to a method of treating a cancer cell overexpressing p185^{Her-2/neu} and fatty acid synthase comprising (a) administering a first anti-cancer therapeutic to an organism comprising the cancer cell, wherein the first anti-cancer therapeutic reduces the activity of p185^{Her-2/neu}; and (b) delivering a fatty acid according to the above-described method to the organism. In preferred embodiments of this aspect of the invention, the anti-cancer effect of the combined treatment is greater than the additive effect of two separate treatments (i.e., the effect is a synergistic effect). This aspect of the invention also comprehends embodiments of the method wherein the organism is a human. Some embodiments of this aspect of the invention comprise a nucleic acid overexpressing p185^{Her-2/neu}, wherein the nucleic acid comprises a promoter comprising a PEA3 binding site. Cancers amenable to treatment by the method include a cancer selected from the group consisting of breast cancer, ovarian cancer, prostate cancer, colorectal cancer, bladder cancer, stomach cancer, lung cancer, oral cancer of the tongue and cancer of the endometrium. Preferred fatty acids for use in this aspect of the invention are unsaturated C16-C22 trans-fatty acids, such as an omega-9 unsaturated fatty acid , an omega-6 unsaturated fatty acid and an omega-3 unsaturated fatty acid, as exemplified by oleic acid, γ-linolenic acid and α-linolenic acid. Any known anti-cancer therapeutic may be used as the first anti-cancer therapeutic, provided it contributes to a lowering of the activity level of p185^{Her-2/neu}. Preferred anti-cancer therapeutics for use as the first anti-cancer therapeutic in the method of the invention are antibodies that specifically recognize p185^{Her-2/neu}, such as trastuzumab.

Another aspect of the invention is drawn to a method of reducing the risk of developing a cancer comprising a cell over-expressing p185^{Her-2/neu} and fatty acid synthase, the method comprising delivering a prophylactically effective amount of a fatty acid as described above. In some embodiments, the method comprises a fatty acid that is an unsaturated C16-C22 trans-fatty acid, such as an omega-9 unsaturated fatty acid, an omega-6 unsaturated fatty acid or an omega-3 unsaturated fatty acid, as exemplified by oleic acid, γ-linolenic acid and α-linolenic acid.

Yet another aspect of the invention is drawn to a kit for treatment of a cancer cell overexpressing p185^{Her-2/neu} and fatty acid synthase comprising a compound that specifically inhibits the binding activity of p185^{Her-2/neu}, a fatty acid and a protocol for the treatment. In some embodiments, the compound that specifically inhibits the binding activity of p185^{Her-2/neu} is an antibody that specifically binds p185^{Her-2/neu}, such as trastuzumab. In some embodiments, the kit comprises a fatty acid that is an unsaturated C16-C22 trans-fatty acid, such as an omega-9 unsaturated fatty acid, an omega-6 unsaturated fatty acid or an omega-3 unsaturated fatty acid, as exemplified by oleic acid, γ-linolenic acid and α-linolenic acid.

Numerous other aspects and advantages of the present invention will be apparent upon consideration of the drawing and detailed description.

### BRIEF DESCRIPTION OF THE DRAWING

FIG. 1. Flow cytometric analysis of cell surface-associated p185^{Her-2/neu} in Her-2/neu-overexpressing BT-474 and SK-Br3 breast cancer cells exogenously supplemented with OA. Overnight serum-starved BT-474 and SK-Br3 breast cancer cells were cultured in Improved Minimum Essential Medium (IMEM; Richter's medium; Biosource International, Camarillo, Calif.) −0.1% FBS in the presence or absence of 20 mM OA for 48 hours. The specific surface expression of p185^{Her-2/neu} in OA-treated cells was determined by flow cytometry by measuring the binding of a mouse anti-p185^{Her-2/neu} monoclonal antibody directed against the extracellular domain of p185^{Her-2/neu} (Ab-5 clone), as described herein. The mean fluorescence signal±SD (n=3) associated with cells for labeled p185^{Her-2/neu} was quantified using the geo mean fluorescence (GM) parameter provided with the Cell Quest Software (Becton Dickinson).

FIG. 2. Exogenous supplementation with OA synergistically enhances trastuzumab-induced down-regulation of p185^{Her-2/neu}. (A) Overnight serum-starved BT-474 breast cancer cells were cultured in IMEM-0.1% FBS supplemented with trastuzumab (top panel), OA (middle panel), or a combination of OA plus trastuzumab (bottom panel) for 48 hours. The amount of cell surface-associated p185^{Her-2/neu} was quantified by flow cytometric analyses using a specific antibody against the extracellular domain of p185^{Her-2/neu} (Ab-5), as described herein. The mean fluorescence signal±SD (n=3) was quantified using the geo mean fluorescence (GM) parameter provided with the Cell Quest Software (Becton Dickinson). (B) Overnight serum-starved BT-474 breast cancer cells were cultured in IMEM-0.1% FBS (panel i) or IMEM-0.1% FBS supplemented with trastuzumab (panel ii), OA (panel iii), or a combination of OA plus trastuzumab (panel iv) for 48 hours in eight-well chamber slides. Cells were then fixed with 4% paraformaldebyde, permeabilized with 0.2% Triton X-100, and labeled for 2 hours with an anti-p185^{Her-2/neu} monoclonal antibody directed against the cytoplasmic domain of p185^{Her-2/neu} (Ab-₃ clone). After labeling, cells were washed thoroughly, and localization of p185^{Her-2/neu} was detected by indirect immunofluorescence by incubating with FITC-conjugated anti-mouse IgG. After counterstaining with DAPI, cells were examined and photographed using a Zeiss fluorescent microscope equipped with a built-in camera. The figure shows a representative immunostaining analysis. Similar results were obtained in three independent experiments. (C) Overnight serum-starved BT-474 breast cancer cells were cultured in IMEM-0.1% FBS supplemented with trastuzumab, OA, or a combination of OA plus trastuzumab for 48 hours, and then harvested and lysed as described herein. Equal amounts of total protein (20 mg per lane) were subjected to Western blot analyses with a specific antibody against p185^{Her-2/neu} (Ab-3 clone), and then re-probed with a β-actin antibody. The figure shows a representative immunoblotting analysis. Similar results were obtained in three independent experiments.

FIG. 3. Exogenous supplementation with OA synergistically enhanced trastuzumab-induced inhibition of breast cancer cell growth in Her-2/neu-overexpressing breast cancer cells. (A) Left panel: exponentially growing SK-Br3 and BT-474 cells were trypsinized and plated in 24-well plates at a density of 10,000 cells/well. Cells were incubated for 24 hours to allow for attachment and starved for serum overnight, after which a zero time point was determined. Cells were treated with OA, trastuzumab, or combinations of these compounds, as specified. Cells were counted at days 0, 3 and 6 with a Coulter Counter. All assays were performed three times in triplicate. The data are presented as mean number of cells×10⁴/well (columns)±SD (bars) after 6 days treatment. Right panels: analyses of the nature of the interaction between the cell growth inhibitory actions of OA and trastuzumab towards SK-Br3 and BT-474 breast cancer cells. For each pair of columns, the height of the columns on the left represents the sum of the effect of each agent alone and, therefore, the expected percentage of cell growth inhibition if their effect was additive when used in combination. The total height of the columns on the right indicates the observed percentage of cell growth inhibition when the agents were used in combination. The difference between the heights of the paired columns reflects the magnitude of synergy of cell growth inhibition (*P<0.05; **P<0.005). (B) Left panels: analysis of the nature of the interaction between the cytotoxic activities of OA and trastuzumab in SK-Br3 and BT-474 breast cancer cells. For each pair of columns, the height of the columns on the left represents the sum of the cytotoxic effect of each agent alone and, therefore, the expected cytotoxicity if their effect was additive when used in combination. The total height of the columns on the right indicates the observed cytotoxicity when the agents were used in combination. The difference between the heights of the paired columns reflects the magnitude of synergy of cytotoxicity (*P<0.05; **P<0.005). Right panels: the combined effect of a simultaneous exposure to OA and trastuzumab was analyzed using the isobologram method, using the IC₅₀ values for SK-Br3 and BT-474 cells. The dashed diagonal line indicates the alignment of theoretical values of an additive interaction between the two compounds. Experimental isoeffect data points at the 50% cytotoxic effect level were generated from the mean survival fractions of four experiments performed in triplicate. Data points above the dashed diagonal line indicative of an additive effect in the isoboles indicates antagonism, while those points below that line of indicated synergy. The values of the mean CI₅₀ values for a particular cell line are also labeled. Student's t-tests were applied to each set of data points to evaluate formally whether synergy (CI<1) or antagonism (CI>1) was evident for a particular cell line as compared with a null-hypothesized I₃₀ of 1 (**P<0.005 versus CI=1, i.e., additivity). (C) Left panels: SK-Br3 and BT-474 cells were plated in soft agarose in the absence (10% FBS) or presence of OA, trastuzumab, or combinations of these compounds, as specified. Colony formation (≧50 μm) was assessed using a colony counter. Each experimental value represents the mean colony number (columns)±SD (bars) from three separate experiments in which triplicate dishes were counted. Right panels: analyses of the nature of the interaction between OA and trastuzumab inhibiting the anchorage-independent colony formation of SK-Br3 and BT-474 breast cancer cells. For each pair of columns, the height of the columns on the left represents the sum of the effect of each agent alone and, therefore, the expected percentage inhibition in colony formation if their effect was additive when used in combination. The total height of the columns on the right indicates the observed percentage inhibition in colony formation when the agents were used in combination. The difference between the heights of the paired columns reflects the magnitude of synergy of cell growth inhibition (*P<0.05; **P<0.005).

FIG. 4. Exogenous supplementation with OA synergistically enhanced trastuzumab-induced inhibition of breast cancer cell viability in Her-2/neu-overexpressing breast cancer cells. See the description of FIG. 3 for experimental protocols and an explanation of data panel presentations.

FIG. 5. Exogenous supplementation with OA synergistically enhanced trastuzumab-induced inhibition of breast cancer cell soft-agar colony formation in Her-2/neu-overexpressing breast cancer cells. See the description of FIG. 3 for experimental protocols and an explanation of data panel presentations.

FIG. 6. Exogenous supplementation with OA synergistically enhanced trastuzumab-induced apoptotic cell death in Her-2/neu-overexpressing breast cancer cells. (A) Top panel: overnight serum-starved BT-474 cells growing in eight-well chamber slides were cultured in IMEM-0.1% FBS in the absence (panel i) or presence (panel ii) of 5 μM OA, 10 μg/ml trastuzumab (panel iii), or a combination of 5 μM OA plus 10 μg/ml trastuzumab (panel iv). After 72 hours, a TUNEL analysis was performed using the DeadEnd® Fluorometric TUNEL System (Promega Inc.) according to the manufacturer's protocol. The immunofluorescence photomicrographs of cells undergoing apoptosis (green staining) and the corresponding DAPI-counterstained photomicrographs are shown. Bottom panel: overnight serum-starved BT-474 breast cancer cells were cultured in IMEM-0.1% FBS in the absence or presence of trastuzumab, OA, or a combination of OA plus trastuzumab for 72 hours, and then harvested and lysed as described herein. Equal amounts of total protein (50 μg per lane) were subjected to Western blot analyses with a specific antibody against the p85 fragment of PARP, and then re-probed with a β-actin antibody. The figure shows a representative immunoblotting analysis. Similar results were obtained in three independent experiments. (B) Analysis of the nature of the interaction between the apoptotic activities of OA and trastuzumab in SK-Br3 and BT-474 breast cancer cells. For each pair of columns, the height of the columns on the left represents the sum of the effect of each agent alone and, therefore, the expected apoptotic cell death if their effect was additive when used in combination. The total height of the columns on the right indicates the observed apoptosis when the agents were used in combination. The difference between the heights of the paired columns reflects the magnitude of synergy of apoptosis (**P<0.005).

FIG. 7. Exogenous supplementation with OA synergistically enhances trastuzumab-induced up-regulation and nuclear accumulation of p₂₇^{Kip1}. Left panels: overnight serum-starved BT-474 breast cancer cells were cultured in IMEM-0.1% FBS in the absence or presence of trastuzumab, OA, or a combination of OA plus trastuzumab for 72 hours, and then harvested and lysed as described herein. Equal amounts of total protein (50 mg per lane) were subjected to Western blot analyses with an anti-p27^{Kip1} rabbit polyclonal antibody and then re-probed with a β-actin antibody. The figure shows a representative immunoblotting analysis. Similar results were obtained in three independent experiments. Right panels: overnight serum-starved BT-474 cells growing in eight-well chamber slides were cultured in IMEM-0.1% FBS in the absence (panel i) or presence of 10 mM OA plus 10 mg/ml trastuzumab (panel ii). After 72 hours, p27^{Kip1} cellular localization was evaluated by immunofluorescence following a 2 hour incubation with an anti-p27^{Kip1} rabbit polyclonal antibody diluted 1:200 in 0.05% Triton X-100/PBS. Cellular localization of P27^{Kip1} was detected by indirect immunofluorescence by incubating with TRITC-conjugated anti-rabbit IgG secondary antibody. Cells were examined and photographed using a Zeiss fluorescent microscope equipped with a built-in camera. The figure shows a representative immunostaining analysis. Similar results were obtained in three independent experiments.

FIG. 8. Exogenous supplementation with OA enhanced trastuzumab-induced inhibition of AKT and MAPK phosphoproteins. Overnight serum-starved BT-474 breast cancer cells were cultured in IMEM-0.1% FBS in the absence or presence of trastuzumab, OA, or a combination of OA plus trastuzumab for 48 hours, and then harvested and lysed as described herein. Equal amounts of total protein (25 mg per lane) were subjected to Western blot analyses with anti-phospho-AKT^{Ser473} or anti-phospho-MAPK antibodies, and then re-probed with anti-AKT, anti-MAPK and β-actin antibodies. The figure shows a representative immunoblotting analysis. Similar results were obtained in three independent experiments.

FIG. 9. γ-Linolenic acid (GLA)-induced PEA3-dependent inhibition of Her-2/neu promoter activity and synergism with trastuzumab. BT-474 (breast cancer), SK-Br3 (breast cancer), MDA-MB-453 (breast cancer), SK-OV3 (ovarian cancer), NCI-N87 (gastrointestinal cancer), and MDA-MB-23 1 (breast cancer) cell lines were obtained from the American Type Culture Collection and routinely grown in phenol red—containing IMEM containing 5% heat-inactivated fetal bovine serum (FBS) and 2 mM L-glutamine. Cells were maintained at 37° C. in a humidified atmosphere of 95% air-5% CO₂. Before experiments, cells were serum-starved overnight and then cultured in IMEM containing 0.1% FBS in the absence or presence of GLA (Sigma Chemical Co., St. Louis, Mo.). GLA was prepared at a concentration of 1 mg/mL in ethanol and was added to a final concentration of 0, 5, 10, or 20 μg/mL; an equal volume of ethanol was added to control cells. Trastuzumab was solubilized in water containing 1.1% benzyl alcohol (stock solution, 21 mg/mL), stored at 4° C., and used within 1 month. A) Cell surface expression of Her-2/neu protein in BT-474 and SK-Br3 cells was determined by flow cytometry using a mouse anti-Her-2/neu antibody directed against the extracellular domain of the p185^{Her-2/neu} oncoprotein (clone Ab-5; Oncogene Research Products; San Diego, Calif.). Briefly, after GLA or control treatment, cells were washed, harvested, resuspended in phosphate-buffered saline (PBS) containing 1% FBS, and incubated with 5 μg/mL Ab-5 antibody for 1 hour at 4° C. The cells were washed again and then incubated with a fluorescein isothiocyanate (FITC)-conjugated anti-mouse IgG secondary antibody (Jackson ImnunoResearch Laboratories, West Grove, Pa.) diluted 1:200 in cold PBS containing 1% FBS for 45 minutes at 4° C. The cells were washed once more in cold PBS, and flow cytometric analysis was performed with a FACScalibur flow cytometer (Becton Dickinson, San Diego, Calif.) equipped with Cell Quest Software (Becton Dickinson). Representative flow cytometry immunofluorescence profiles in untreated control cells (blue line) and in GLA-treated cells (orange line) are shown. As a control for nonspecific immunofluorescence, cells were stained with the secondary antibody alone (solid black line). The mean fluorescence signal associated with cells for labeled p185^{Her-2/neu} was quantified using the Geo Mean (GM) fluorescence parameter provided with the software. Data are the means and 95% confidence intervals (CIs) of three independent experiments. B) Immunoblot analysis of Her-2/neu and PEA3 proteins in control- and GLA-treated BT-474 and SK-Br3 cells. Cells were washed twice with PBS and then lysed in buffer (20 m M Tris [pH 7.5], 150 m M NaCl, 1 mM EDTA, 1 m M EGTA, 1% Triton X-100, 2.5 m M sodium pyrophosphate, 1 mM β-glycerolphosphate, 1 mM Na3VO4, 1 μg/ml leupeptin, 1 mM phenylmethylsulfonyl fluoride) for 30 minutes on ice. Equal amounts of protein (10 μg) were heated in sodium dodecyl sulfate (SDS) sample buffer for 10 minutes at 70° C., subjected to electrophoresis on either 3-8% NuPAGE Tris-acetate (p185^{Her-2/neu}) or 10% SDS-polyacrylamide gel electrophoresis (PEA3) gels, and transferred to nitrocellulose membranes. Nonspecific binding was blocked by incubation for 1 hour with TBS-T (25 mM Tris-HCl [pH 7.5], 150 mM NaCl, and 0.05% Tween-20) containing 5% nonfat dry milk. The treated filters were washed in TBS-T and then incubated with primary antibodies, anti-p185^{Her-2/neu} mouse monoclonal antibody—Clone Ab-3 [Oncogene Research Products, San Diego, Calif.] and anti-PEA3 mouse monoclonal antibody—sc-113 [Santa Cruz Biotechnology, Santa Cruz, Calif.], for 2 hours in TBS-T containing 5% (w/v) nonfat dry milk. The membranes were washed in TBS-T and horseradish peroxidase—conjugated secondary antibodies in TBS-T were added for 45 minutes. Immunoreactive bands were detected by enhanced chemiluminescence reagent (Pierce, Rockford, Ill.). Blots were reprobed with an anti-β-actin goat polyclonal antibody (Santa Cruz Biotechnology) to control for protein loading and transfer. Densitometric values of protein bands were quantified using Scion Imaging Software (Scion Corp., Frederick, Md.). Representative blots are shown; similar results were obtained in three independent experiments. C) Reverse transcription (RT)—polymerase chain reaction (PCR) analysis of Her-2/neu expression in BT-474 and SK-Br3 breast cancer cells. Total RNA from control-treated BT-474 and SK-Br3 cells or cells treated with varying amounts of GLA (0, 5, 10, or 20 μg/mL) was extracted with the TriPure Isolation Reagent (Boehringer-Mannheim). One microgram of total RNA was reverse-transcribed and amplified with the Access RT-PCR System (Promega Inc.) using 1 mM of specific primers for Her-2/neu (sense: 5′-GGGCTGG CCC GATGTATTTGAT-3′; SEQ ID NO: 1: antisense: 5′-ATAGAGGTTGTCGAAGGCTGGGC-3′; SEQ ID NO: 2). As an internal control, β-actin primers were used. The RT reaction was carried out for 45 minutes at 48° C. Her-2/neu and β-actin complementary DNAs were amplified for 20 cycles of the following conditions: 96° C. for 30 seconds, 60° C. for 1 minute, and 68° C. for 2 minutes. The PCR products were separated on 2% agarose gels and detected by ethidium bromide staining. Results are representative of three independent experiments. D) MDA-MB-453, SK-OV3, NCI-N87, and MDA-MB-231 cells were treated for 48 hours with either ethanol or GLA (10 μg/mL) and then subjected to immunoblot analysis of Her-2/neu, PEA3, and β-actin (as a control). Representative blots from three independent experiments are shown. E) Apoptosis was analyzed by terminal deoxynucleotidyltransferase-mediated dUTP-biotin nick end labeling (TUNEL) analysis using the DeadEnd Fluorometric TUNEL System (Promega Inc.). Briefly, BT-474 cells were split at a density of 2×10⁴ cells/well in eight-well Lab-Tek chamber slides. After 24 hours, the cells were treated with trastuzumab (10 μg/mL) and/or GLA (10 μg/mL) for 48 hours. Cells were then washed twice with PBS, fixed in 4% methanol-free paraformaldehyde for 10 minutes, washed twice with PBS, and permeabilized with 0.2% Triton X-100 for 5 minutes. After two more washes, each slide was covered with equilibration buffer for an additional 10 minutes. The buffer was then aspirated, and the slides were incubated with terminal deoxynucleotidyl transferase buffer at 37° C. for 1 hours. The reaction was stopped with 2×standard saline citrate, washed with PBS and mounted with Vectashield+4′, 6-diamidino-2-phenylindole (DAPI) (Vector Laboratories, Burlingame, Calif.). Representative immunofluorescence photographs of cells undergoing apoptosis (green TUNEL staining) and the corresponding DAPI-counterstained photomicrographs are shown in the top panel. Apoptosis was quantified by determining the percentage of BT-474 and SK-Br3 cells containing nuclei with complete TUNEL-associated staining per total cells, as determined by DAPI staining of four random fields (bottom panel). For each pair of columns, the height of the columns on the left represents the sum of the effects of each agent alone, and the total height of the columns on the right indicates the observed apoptosis when the agents were used in combination. Data are the means and 95% confidence intervals of four independent experiments. One-factor analysis of variance was used to analyze differences in the percentage of apoptosis between the various treatment groups and the control group. *Two-sided P< .001 for the GLA+trastuzumab group versus all other groups. All statistical tests were two-sided.

FIG. 10. Effect of GLA treatment on the sensitivity of Her-2/neu-overexpressing breast cancer cells to trastuzumab. A) Trastuzumab sensitivity was determined using a standard colorimetric 3,4,5-dimethylthiazol-2-yl-2,5-diphenyl-tetrazolium bromide(MTT) reduction assay. Cells in exponential growth were harvested, seeded at a concentration of ˜5×10³ cells/200 μl/well into 96-well plates and allowed to attach overnight. The medium was replaced, and various concentrations of trastuzumab, γ-linolenic acid (GLA), or combinations of compounds were added. Agents were studied in combination concurrently, and they were not renewed during the entire period of cell exposure. After treatment, the medium was removed and replaced by fresh drug-free medium (100 μl/well), and MTT (5 mg/ml in phosphate-buffered saline [PBS]) was added to each well at a 1/10 volume. After 2-3 hours at 37° C., the supernatants were removed, 100 μl of dimethylsulfoxide was added to each well, and the plates were agitated. Absorbances were measured at 570 nm using a multiwell plate reader (Model Anthos Labtec 2010 1.7 reader). The cell viability effects from exposure of cells to each agent alone and to their combination were analyzed by generating concentration-effect curves as a plot of the fraction of unaffected (surviving) cells versus drug concentration. Dose-response curves were plotted as percentages of the control cell absorbances, which were obtained from control wells treated with appropriate concentrations of vehicles that were processed simultaneously. For each treatment, cell viability was evaluated as a percentage using the following equation: (A₅₇₀ of treated sample/A₅₇₀ of untreated sample)×100. Drug sensitivity was expressed in terms of the concentration of drug required for 50% (IC₅₀) reduction of cell viability. Because the percentage of control absorbance was considered to be the surviving fraction of cells, the IC₅₀ values were defined as the concentration of drug that produced 50% reduction in control absorbance (by interpolation). The degree of sensitization to trastuzumab by GLA was evaluated by dividing the IC₅₀ values of control cells by those obtained when cells were co-exposed to GLA during treatment with trastuzumab. Data are the mean (columns) and 95% confidence intervals (bars) of four experiments performed in triplicate. One-factor analysis of variance (ANOVA) was used to analyze differences in the levels of sensitivity to trastuzumab as measured by MTT-based determination of IC₅₀ values between the co-treatment group (i.e., GLA+trastuzumab) and the control (i.e., trastuzumab) group. *P< .001 compared with the control (untreated) group (one-factor ANOVA). All statistical tests were two-sided. B) Isobologram analysis of the combined cytotoxic effect of simultaneous exposure to GLA and trastuzumab. The concentration of trastuzumab producing a desired (e.g., 50% inhibitory) effect was plotted on the x-axis, and the concentration of GLA producing the same degree of effect was plotted on they-axis; a straight line joining these two points represents zero interaction (addition) between two agents. The experimental isoeffect points are the concentrations (expressed relative to the IC₅₀ concentrations) of the two agents which, when combined, kill 50% of the cells. When the experimental isoeffect points fall below the addition line, the combination effects are considered to be supra-additive or synergistic, whereas antagonism occurs if the points lie above it. C and D) Analysis of soft-agar colony formation in Her-2/neu overexpressing breast cancer cells. The efficiency of colony formation in liquid culture was determined by monitoring anchorage-independent cell growth in soft-agar experiments (C). A bottom layer of 1 mL of IMEM containing 0.6% agar and 10% fetal bovine serum (FBS) was prepared in 35-mm multiwell cluster dishes. Cells (10⁴/dish) were added in a 1 mL top layer containing GLA, trastuzumab, a combination of GLA plus trastuzumab, or vehicle in 0.35% agar and 10% FBS, as specified. Dishes were incubated in a humidified 5% CO₂ incubator at 37° C., and colonies (at least 50 μm) were counted after approximately 14 days following staining with nitroblue tetrazolium (Sigma Chemical Co., St. Louis, Mo.). Each experimental value represents the mean colony number (columns) and 95% confidence intervals (bars) from three separate experiments in which triplicate dishes were counted. One-factor ANOVA was used to analyze differences in the number of colonies among the various treatment groups and the control group. *P<0.001 for the GLA+trastuzumab groups versus all other groups (one-factor ANOVA). All statistical tests were two-sided. For each pair of columns in D, the height of the columns on the left represents the sum of the effect of each agent alone and, therefore, the expected percent inhibition in colony formation if their effect was additive when used in combination. The total height of the columns on the right indicates the observed percent inhibition in colony formation when the agents were used in combination. The difference between the heights of the paired columns reflects the magnitude of the synergy on reducing soft-agar colony formation. Data are the means (columns) and 95% confidence intervals (bars) of three experiments performed in triplicate. One-factor ANOVA was used to analyze differences in the percentages of reduction in soft-agar colony formation number among the various treatment groups. *P<0.001 for the GLA+trastuzumab groups versus all other groups (one-factor ANOVA). All statistical tests were two-sided.

FIG. 11. Luciferase activity in transiently transfected cells. Luciferase activity was assayed in cells that were transiently transfected with a pGL2-Luc construct containing a luciferase reporter gene under the control of a Her-2/neu promoter fragment containing a wild-type (top panel, left) or mutant (top panel, right) PEA3 binding site as described herein. The magnitude of activation in Her-2/neu promoter-luciferase-transfected cells was determined after normalization of the luciferase activity obtained in cells co-transfected with equivalent amounts of the empty pGL2-Luciferase vector lacking the Her-2/neu promoter (-luciferase) and the internal control plasmid pRL-CMV. This control value was used to calculate the relative change in the transcriptional activities of Her-2/neu-promoter-luciferase-transfected cells in response to treatments after normalization to pRL-CMV. The activity of the wild-type promoter in untreated control cells was defined as 100%. The activity of the mutated promoter in untreated control cells was calculated relative to that found in untreated control cells transfected with the intact (Her-2/neu wild-type PEA3-binding site-luciferase) Her-2/neu promoter (=100%). Data are the mean and 95% confidence intervals (95% CI) of three experiments performed in triplicate. One-factor ANOVA was used to analyzed differences in the percentages of luciferase activity between the various treatment groups.

FIG. 12. Immunoblot analyses of Her-2/neu and PEA3 proteins in control- and OA-treated cancer cells. SK-Br3, SK-OV3, NCI-N87, MCF-7/neo and MCF-7/Her2-18 cells were treated for 48 hours with either ethanol (v/v) or OA (20 μM) and then subjected to immunoblot analysis of Her-2/neu, PEA3 and β-actin (as control) as described herein. Representative blots from three independent experiments are shown.

FIG. 13. Model for OA-induced transcriptional repression of Her-2/neu oncogene in cancer cells. a. Features of the Her-2/neu promoter. The Her-2/neu promoter from −75 to +15 is represented, with an additional area illustrating sequences upstream of −200. The major (+1 bp) and minor (−69 bp) transcription start sites are indicated with arrows and the positions of the TATA (−22 to −26 bp) and CCAAT (−71 to −75 bp) boxes are marked. The relative positions of the main transcription factor binding sites, AP-2, Ets and ZONAB, are indicated, with the sequences below each giving the core binding site defined for each factor (modified from {31}, incorporated herein by reference). Mutation of the Ets binding site (EBS; 5′-GAGGAA-3′; see SEQ ID NOS: 3 and 4), at −33 to −28, impairs reporter activity {35}, while it has also been reported that binding of Ets factors to the EBS induces a severe bend in the DNA {48}. At least 10 different Ets proteins have been found in cancer cells at varying levels but, of those, only PEA3 has so far been shown to correlate in distribution with Her-2/neu overexpression. It is likely that if the EBS is occupied by PEA3, then the TATA-binding protein will not be able to access the closely associated TATA box, thus repressing the Her-2/neu promoter. b. Cancer cells expressing physiological levels of Her-2/neu naturally exhibit high levels of the trans-repressor PEA3 and constitutively low transcriptional activity of the Her-2/neu gene promoter. In this scenario, exogenous supplementation with OA does not modulate PEA3 expression and, therefore, Her-2/neu gene promoter activity continues to be inhibited by PEA3. c. Cancer cells bearing Her-2/neu gene amplification naturally express low to undetectable levels of the trans-repressor PEA3 and, therefore, a PEA3 binding site-enhanced transcriptional activity of the Her-2/neu gene promoter. Exogenous supplementation with OA promotes accumulation of the trans-repressor PEA3 and, hence, occupation of the PEA3 binding site. OA-induced formation of inhibitory “PEA3-PEA3 DNA binding site” complexes at the Her-2/neu gene promoter in Her-2/neu gene-amplified cancer cells operates equally in various types of human malignancies.

### DETAILED DESCRIPTION

The present invention provides materials and methods useful alone or in combination with therapeutics/therapies in the treatment or prevention of a variety of cancers characterized by cancer cells overexpressing p185^{Her-2/neu} (i.e., Her-2/neu) and exhibiting elevated levels of fatty acid synthase (i.e., FAS). The materials of the invention are kits comprising fatty acids, such as purified forms of naturally occurring unsaturated C16-C22 trans-fatty acids. Preferred forms of the fatty acids include monounsaturated ω-9 fatty acids (e.g., oleic acid), polyunsaturated ω-6 fatty acids (e.g., γ-linolenic acid), and polyunsaturated ω-3 fatty acids (e.g., α-linolenic acid). Suitable cancers include any cancer characterized by cancer cells overexpressing p185^{Her-2/neu} and having elevated FAS activity; including breast cancer, ovarian cancer, prostate cancer, colorectal cancer, bladder cancer, stomach cancer, lung cancer, oral cancer of the tongue and cancer of the endometrium.

The invention comprehends use of the materials of the invention alone or in combination with any known anti-cancer treatment. By way of exemplifying a combined treatment method, a therapeutically effective amount of a fatty acid according to the invention is administered before, after, or concomitantly with, an antibody specifically recognizing or binding to p185^{Her-2/neu}, such as trastuzumab.

The results disclosed herein indicate that ω-6 mono-unsaturated fatty acids, exemplified by trans-18:1n-9 (i.e., oleic acid or OA), ω-6 polyunsaturated fatty acids, exemplified by trans 18:3 n-6 (i.e., γ-linolenic acid or GLA), specifically suppress Her-2/neu overexpression which, in turn, interacts synergistically with anti-Her-2/neu breast cancer immunotherapy by promoting apoptotic cell death of breast cancer cells with an amplification of the Her-2/neu oncogene. See Table 1 for identification and characterization of fatty acids.

TABLE 1
Fatty Acids
Chemical Names and Descriptions of Some Common Fatty Acids

| | | | | |
|---|---|---|---|---|
| Butyric Acid | 4 | 0 | butanoic acid | butterfat |
| Caproic Acid | 6 | 0 | hexanoic acid | butterfat |
| Caprylic Acid | 8 | 0 | octanoic acid | coconut oil |
| Capric Acid | 10 | 0 | decanoic acid | coconut oil |
| Lauric Acid | 12 | 0 | dodecanoic acid | coconut oil |
| Myristic Acid | 14 | 0 | tetradecanoic acid | palm kernel oil |
| Palmitic Acid | 16 | 0 | hexadecanoic acid | palm oil |
| Palmitoleic Acid | 16 | 1 | 9-hexadecenoic acid | animal fats |
| Stearic Acid | 18 | 0 | octadecanoic acid | animal fats |
| Oleic Acid | 18 | 1 | 9-octadecenoic acid | olive oil |
| Vaccenic Acid | 18 | 1 | 11-octadecenoic acid | butterfat |
| Linoleic Acid | 18 | 2 | 9,12-octadecadienoic acid | grape seed oil |
| Alpha-Linolenic Acid (ALA) | 18 | 3 | 9,12,15-octadecatrienoic acid | flaxseed (linseed) oil |
| Gamma-Linolenic Acid (GLA) | 18 | 3 | 6,9,12-octadecatrienoic acid | borage oil |
| Arachidic Acid | 20 | 0 | eicosanoic acid | peanut oil, fish oil |
| Gadoleic Acid | 20 | 1 | 9-eicosenoic acid | fish oil |
| Arachidonic Acid (AA) | 20 | 4 | 5,8,11,15-eicosatetraenoic acid | liver fats |
| EPA | 20 | 5 | 5,8,11,14,17-eicosapentaenoic acid | fish oil |
| Behenic acid | 22 | 0 | docosanoic acid | rapeseed oil |
| Erucic acid | 22 | 1 | 13-docosenoic acid | rapeseed oil |
| DHA | 22 | 6 | 4,7,10,13,16,19-docosahexaenoic acid | fish oil |
| Lignoceric acid | 24 | 0 | tetracosanoic acid | small amounts in most fats |

Her-2/neu (erbB-2) is one the most commonly analyzed oncogenes in breast cancer studies. This tyrosine kinase receptor regulates biological functions as diverse as cellular proliferation, transformation, differentiation, motility and apoptosis [52]. Therefore, modulation of Her-2/neu expression must be tightly regulated for normal cellular function. Consistent with this view, in vitro and animal studies demonstrate that deregulated Her-2/neu overexpression plays a pivotal role in oncogenic transformation, tumorigenesis and metastasis.

Her-2/neu overexpression occurs in about 20% of breast carcinomas and is associated with unfavorable clinical outcome and resistance to chemotherapy [53-56]. Little is known about the ultimate biochemical pathways through which fatty acids such as OA influence breast cancer risk and/or breast cancer progression. The results disclosed herein demonstrate that fatty acids (e.g., OA) can suppress Her-2/neu oncogene overexpression, representing a novel pathway through which individual dietary fatty acids modulate both the etiology and the aggressive behavior of cancer, such as breast cancer.

No toxicities have been reported or suspected with fatty acids such as OA consistent with use of one or more fatty acids as a dietary supplement, thereby providing a promising dietary intervention for the prevention and/or management of Her-2/neu-overexpressing carcinomas. Moreover, the data disclosed herein indicate further that dietary interventions based on, e.g., OA may be even more beneficial when given in combination with other therapies directed against Her-2/neu. Thus, OA co-exposure induces a dramatic increase in the sensitivity of Her-2/neu-overexpressing breast cancer cells to trastuzumab-induced cell growth inhibition upon anchorage-dependent and -independent conditions, and the nature of the interaction between OA and trastuzumab was found to be synergistic at clinically relevant trastuzumab concentrations. Importantly, exogenous supplementation with OA synergistically enhanced the ability of trastuzumab to induce down-regulation of p185^{Her-2/neu}. Even without amplification of the Her-2/neu gene, moreover, the concurrent exposure to OA and trastuzumab was synergistically cytotoxic towards Her-2/neu-overexpressing cells by promoting DNA fragmentation associated with apoptotic cell death, as confirmed by TUNEL staining and cleavage of the caspase-3 substrate, PARP. The sensitizing effects of OA on trastuzumab efficacy were also accompanied by the up-regulation and nuclear accumulation of p27^{Kip1}, a cyclin-dependent kinase inhibitor that plays a key role in the onset and progression of Her-2/neu-induced breast tumorigenesis that has recently been implicated in the development of trastuzumab resistance in breast cancer cells [42-44, 49-51]). Additionally, exogenous supplementation with OA significantly enhanced the ability of trastuzumab to inhibit the signaling pathways downstream of Her-2/neu that regulate cell cycle progression and/or cell death (i.e., AKT and MAPK).

As described in the following examples, flow cytometry, Western blotting, immunofluorescence microscopy, metabolic status (MTT), soft-agar colony formation, enzymatic in situ labeling of apoptosis-induced DNA double-strand breaks (TUNEL assay analyses), and caspase-3-dependent poly-ADP ribose polymerase (PARP) cleavage assays were used to characterize the effects of exogenous supplementation with a fatty acid of OA on the expression of the Her-2/neu oncogene, which plays an active role in breast cancer etiology and progression. In addition, the effects of administering a fatty acid (e.g., OA) on the efficacy of trastuzumab (Herceptin®), a humanized monoclonal antibody binding with high affinity to the ectodomain of the Her-2/neu-encoded p185^{Her-2/neu} oncoprotein, were investigated. To study these issues, BT-474 and SKBr-3 breast cancer cells, which naturally exhibit amplification of the Her-2/neu oncogene, were used.

Flow cytometric analyses demonstrated a dramatic (up to 46%) reduction of cell surface-associated p185^{Her-2/neu} following treatment of the Her-2/neu-overexpressing BT-474 and SK-Br3 cell lines with OA. This effect was comparable to that found following exposure to optimal concentrations of trastuzumab (up to 48% reduction with 20 mg/ml trastuzumab). Importantly, OA-induced suppression of Her-2/neu overexpression was not significantly prevented by the effective scavenger of reactive oxygen species vitamin E, thus ruling out that lipid peroxidation may be involved in this effect. Remarkably, the concurrent exposure to OA and suboptimal concentrations of trastuzumab (5 mg/ml) synergistically down-regulated Her-2/neu expression, as determined by flow cytometry (up to 70% reduction), immunoblotting, and immunofluorescence microscopy studies. The nature of the cytotoxic interaction between OA and trastuzumab revealed a strong synergism, as assessed by MTT-based cell viability and anchorage-independent soft-agar colony formation assays. Moreover, OA co-exposure synergistically enhanced trastuzumab efficacy towards Her-2/neu overexpressing cells by promoting DNA fragmentation associated with apoptotic cell death, as confirmed by TUNEL and caspase-3-dependent PARP cleavage. In addition, treatment with OA and trastuzumab dramatically increased both the expression and the nuclear accumulation of p27^{Kip1}, a cyclin-dependent kinase inhibitor playing a key role in the onset and progression of Her-2/neu-related breast cancer. OA co-exposure also significantly enhanced the ability of trastuzumab to inhibit signaling pathways downstream of Her-2/neu, including phosphoproteins such as AKT and MAPK. Results indicate that OA is transcriptionally repressing Her-2/neu expression by up-regulating PEA3, an ets DNA-binding protein that inhibits Her-2/neu-promoted tumorigenesis by down-regulating Her-2/neu promoter activity [58-60]. These findings demonstrate that OA, the main mono-unsaturated fatty acid of olive oil, suppresses Her-2/neu overexpression which, in turn, interacts synergistically with anti-Her-2/neu immunotherapy by promoting the apoptotic cell death of breast cancer cells with Her-2/neu oncogene amplification. This previously unrecognized property of non-toxic fatty acids such as OA provides a molecular mechanism by which individual fatty acids regulate the malignant behavior of breast cancer cells, thereby providing methods for reducing the risk of developing any of a variety of erbB-2-overexpressing breast cancers, methods of treating such cancers, methods of maintaining the treatment of such cancers, and methods of mitigating or alleviating at least one symptom associated with such a cancer.

### EXAMPLE 1

### Material and Methods

Cell Lines and Culture Conditions

The human breast cancer cell lines SK-Br3 and BT-474 were obtained from the American Type Culture Collection (ATCC), and they were routinely grown in phenol red-containing improved modified essential medium (IMEM; Biosource International, Camarillo, Calif.) containing 5% (v/v) heat-inactivated fetal bovine serum (FBS) and 2 mM L-glutamine. Cells were maintained at 37° C. in a humidified atmosphere of 95% air/5% CO₂. Cells were screened periodically for Mycoplasma contamination.

Oleic acid (18:1n-9) and vitamin E (dl-α-tocopherol) were purchased from Sigma Chemical Co. (St Louis, Mo.). The cultures were supplemented, where indicated, with fatty acid-free bovine serum albumin (FA-free BSA; 0.1 mg/ml) complexed with a specific concentration of OA. A BSA-OA concentrate was formed by mixing 1 ml BSA (10 mg/ml) with various volumes (1-10 ml) of OA (200 mg/ml) in ethanol. The concentrate was mixed for 30 minutes at room temperature before addition to the cultures. Control cultures contained uncomplexed BSA. Trastuzumab (Herceptin®) is commercially available from Genentech, Inc.

The mouse monoclonal antibodies for p185^{Her-2/neu} (Ab-3 and Ab-5 clones) were from Oncogene Research Products (San Diego, Calif.). Anti-β-actin goat polyclonal and anti-p27^{Kip1} rabbit polyclonal antibodies were from Santa Cruz Biotechnology (Santa Cruz, Calif.). The anti-PARP p85 fragment antibody was from Promega Corp. (Madison, Wis.). Anti-MAPK, anti-phospho-MAPK, anti-AKT and anti-phospho-AKT^{Ser473} rabbit polyclonal antibodies were from Cell Signal Technology (Beverly, Md.).

Flow Cytometry

Cells were seeded on 100-mm plates and cultured in complete growth medium. Upon reaching 75% confluence, the cells were washed twice with pre-warmed PBS and cultured in serum-free medium overnight. OA, trastuzumab, or a combination of OA plus trastuzumab as specified was added to the culture as specified, and incubation was carried out at 37° C. up to 48 hours in low-serum (0.1% FBS) medium. After treatment, cells were washed once with cold PBS and harvested in cold PBS. The cells were pelleted and resuspended in cold PBS containing 1% FBS. The cells were then incubated with an anti-p185^{Her-2/neu} mouse monoclonal antibody (clone Ab-5) at 5 μg/ml for 1 hour at 4° C. The cells were then washed twice with cold PBS, resuspended in cold PBS containing 1% FBS, and incubated with a fluorescein isothiocyanate (FITC)-conjugated anti-mouse IgG secondary antibody (Jackson Immunoresearch Laboratories, West Grove, Pa.) diluted 1:200 in cold PBS containing 1% FBS for 45 minutes at 48° C. Finally, the cells were washed once in cold PBS, and flow cytometric analysis was performed using a FACScalibur flow cytometer (Becton Dickinson, San Diego, Calif.) equipped with Cell Quest Software (Becton Dickinson). The mean fluorescence signal associated with cells for labeled p185^{Her-2/neu} was quantified using the GEO MEAN fluorescence parameter provided with the software.

Immunoblotting

Following treatments with OA, trastuzumab, or a combination of OA plus trastuzumab, as specified, cells were washed twice with PBS and then lysed in buffer [20 mM Tris (pH 7.5), 150 mM NaCl, 1 mM EDTA, 1 mM EGTA, 1% Triton X-100, 2.5 mM sodium pyrophosphate, 1 mM B-glycer-olphosphate, 1 mM Na3VO4, 1 mg/ml leupeptin, 1 mM phenylmethylsulfonylfluoride] for 30 minutes on ice. The lysates were cleared by centrifugation in an Eppendorf tube (15 minutes at 14,000 r.p.m. at 4° C.). Protein content was determined against a standardized control using the Pierce protein assay kit (Rockford, Ill.). Equal amounts of protein were heated in SDS sample buffer (Laemli buffer) for 10 minutes at 70° C., subjected to electrophoresis on either 3-8% NuPAGE or 10% SDS-PAGE (p27^{Kip1}, ^{PARP}, MAPK and AKT), and then transferred to nitrocellulose membranes. Non-specific binding on the nitrocellulose filter paper was minimized by blocking for 1 hour at room temperature (RT) with TBS-T [25 mM Tris-HCl, 150 mM NaCl (pH 7.5) and 0.05% Tween 20] containing 5% (w/v) non-fat dry milk. The treated filters were washed in TBS-T and then incubated overnight at 4° C. with specific primary antibodies in TBS-T/5% (w/v) BSA. The membranes were washed in TBS-T, horseradish peroxidase-conjugated secondary antibodies (Jackson Immunoresearch Laboratories) in TBS-T were added for 1 hour, and immunoreactive bands were detected by enhanced chemiluminescence reagent (Pierce). Blots were re-probed with an antibody for B-actin to control for protein loading and transfer. Densitometric values of protein bands were quantified using Scion Imaging Software (Scion Corp., Frederick, Md.).

In Situ Immunofluorescent Staining

Cells were seeded at a density of 1×10⁴ cells/well in a four-well chamber slide (Nalge Nunc International, Rochester, N.Y.). Following treatments with OA, trastuzumab, or a combination of OA plus trastuzumab, as specified, cells were washed with PBS, fixed with 4% paraformaldehyde in PBS for 10 minutes, permeabilized with 0.2% Triton X-100/PBS for 15 minutes, and stored overnight at 4° C. with 10% horse serum in PBS. The cells were washed and then incubated for 2 hours with anti-p185^{Her-2/neu} or anti-p27^{Kip1} antibodies, each separately diluted 1:200 in 0.05% Triton X-100/PBS. After extensive washes, the cells were incubated for 45 minutes with FITC-conjugated anti-mouse IgG (p185^{Her-2/neu}) or tetramethylrhodamine isothiocyanate (TRITC)-conjugated anti-rabbit IgG (p27^{Kip1}), each again separately diluted 1:200 in 0.05% Triton X-100/PBS. The cells were washed five times with PBS and mounted with VECTASHIELD+DAPI (Vector Laboratories, Burlingame, Calif.). As controls, cells were stained with primary or secondary antibody alone. Control experiments did not display significant fluorescence in any case. Indirect immunofluorescence was recorded on a Zeiss microscope. Images were noise-filtered, corrected for background and prepared using Adobe Photoshop.

Anchorage-dependent Cell Proliferation

SK-Br3 and BT-474 cells exponentially growing in IMEM-5% FBS were trypsinized and re-plated in 24-well plates at a density of 10,000 cells/well. Cells were incubated for 24 hours to allow for attachment, after which a zero time point was determined. Cells were treated with OA, trastuzumab, or a combination of OA plus trastuzumab, as specified. Cell number was determined at days 0, 3 and 6 with a Coulter Counter (Coulter Electronics, Inc., Hialeah, Fla.). All assays were performed at least three times in triplicate. The data are presented as mean of number cells 10⁴/well±SD.

In Vitro Chemosensitivity Testing

Trastuzumab sensitivity was determined using a standard colorimetric MTT (3-4,5-dimethylthiazol-2-yl-2,5-diphenyl-tetrazolium bromide) reduction assay. Cells in exponential growth were harvested by trypsinization and seeded at a concentration of 5×10³ cells/200 μl/well into 96-well plates, and incubated overnight for attachment. The medium was then removed and fresh medium, along with various concentrations of trastuzumab, OA or combinations of compounds, was added to cultures in parallel. Agents were studied in combination concurrently. Control cells without agents were cultured using the same conditions with comparable media changes. Compounds were not renewed during the entire period of cell exposure. Following treatment, the medium was removed and replaced with fresh drug-free medium (100 μl/well), and MTT (5 mg/ml in PBS) was added to each well at a volume of 1:10. After incubation for 2-3 hours at 37° C., the supernatants were carefully aspirated, 100 ml of DMSO were added to each well, and the plates were agitated to dissolve the crystal product. Absorbances were measured at 570 nm using a multi-well plate reader (Model Anthos Labtec 2010 1.7 reader). The cell viability effects from exposure of cells to each compound alone and to their combination were analyzed, generating concentration-effect curves as a plot of the fraction of unaffected (surviving) cells versus drug concentration. Dose-response curves were plotted as percentages of the control cell absorbances, which were obtained from control wells treated with appropriate concentrations of the compound vehicles that were processed simultaneously. For each treatment, cell viability was evaluated as a percentage using the following equation: (A570 of treated sample/A570 of untreated sample)×100. Drug sensitivity was expressed in terms of the concentration of drug required for a 50% reduction of cell viability (IC₅₀). Since the percentage of control absorbance was considered to be the surviving fraction of cells, the IC₅₀ values were defined as the concentration of drug that produced a 50% reduction in control absorbance (by interpolation). The degree of sensitization to trastuzumab by OA was evaluated by dividing IC₅₀ values of control cells by those obtained when cells were exposed to OA during exposure to trastuzumab.

Determination of Synergism: Isobologram Analysis

The interaction between OA and trastuzumab was evaluated using the isobologram technique [45], a dose-oriented geometric method of assessing drug interactions. With the isobologram method, the concentration of one agent producing a desired (e.g. 50% inhibitory) effect is plotted on the horizontal axis and the concentration of another agent producing the same degree of effect is plotted on the vertical axis. A straight line joining these two points represents zero interaction (addition) between two agents. The experimental isoeffect points are the concentrations (expressed relative to the IC₅₀ concentrations) of the two agents that, when combined, kill 50% of the cells. When the experimental isoeffect points fall below that line, the combination effect of the two drugs is considered to be supra-additive or synergistic, whereas antagonism occurs if the point lies above the line. A quantitative index of these interactions was provided by the isobologram equation: CIₓ=(a/A)+(b/B), where, for this study, A and B represent the respective concentrations of OA and trastuzumab required to produce a fixed level of inhibition (IC₅₀) when administered alone, a and b represent the concentrations required for the same effect when the drugs were administered in combination, and CIx represents an index of drug interaction (interaction index). Ix values<1 indicate synergy, a value of 1 represents addition, and values of>1 indicate antagonism.

Soft-agar Colony-formation Assays

The efficiency of colony formation in liquid culture was determined by monitoring anchorage-independent cell growth in soft-agar experiments. A bottom layer of 1 ml IMEM containing 0.6% agar and 10% FBS was prepared in 35-mm multi-well cluster dishes. After the bottom layer solidified, cells (10,000/dish) were added in a 1 ml top layer containing OA, trastuzumab, a combination of OA plus trastuzumab, or vehicles (v/v) in 0.35% agar and 10% FBS, as specified. All samples were prepared in triplicate. Dishes were incubated in a humidified 5% CO₂ incubator at 37° C., and colonies measuring >50 mm were counted 14 days after staining with nitroblue tetrazolium (Sigma) using a cell colony counter (Ommias 3600; Imaging Products International, Inc., Charley, Va.).

Apoptosis

Detection of apoptosis in SK-Br3 and BT-474 cells treated with OA, trastuzumab, or a combination of OA plus trastuzumab, as specified, was performed by terminal deoxynucleotidyltransferase-mediated dUTP-biotin nick-end labeling (TUNEL) analysis using the DeadEnd® Fluorometric TUNEL System (Promega Inc.) according to the manufacturer's instructions. Briefly, cells were split at a density of 2×10⁴ cells/well in an eight-well chamber slide (Lab-Tek). After 48 hours incubation the cells were treated with trastuzumab in the absence or presence of OA for 72 hours. Following treatment, cells were washed twice with PBS and fixed with 4% methanol-free paraformaldehyde for 10 minutes. Cells were washed twice more with PBS and permeabilized with 0.2% Triton X-100 for 5 minutes. After two more washes, each slide was covered with equilibration buffer for 10 minutes. The buffer was then aspirated, and the slides were incubated with TdT buffer at 37° C. for 1 hour. The reaction was stopped with 2×standard saline citrate and the slides were viewed under an immunofluorescence microscope (Zeiss). Apoptosis was quantified by determining the proportion of cells containing nuclei with complete TUNEL-associated staining. One hundred cells were assessed in triplicate for each treatment.

Statistical Analysis

Statistical analysis of mean values was performed using the nonparametric Mann-Whitney test. Differences were considered significant at P<0.05 and P<0.005.

### EXAMPLE 2

### Exogenous Supplementation with a Fatty Acid Down-regulates Her-2/neu in Her-2/neu-overexpressing BT-474 and SK-Br3 Breast Cancer Cells

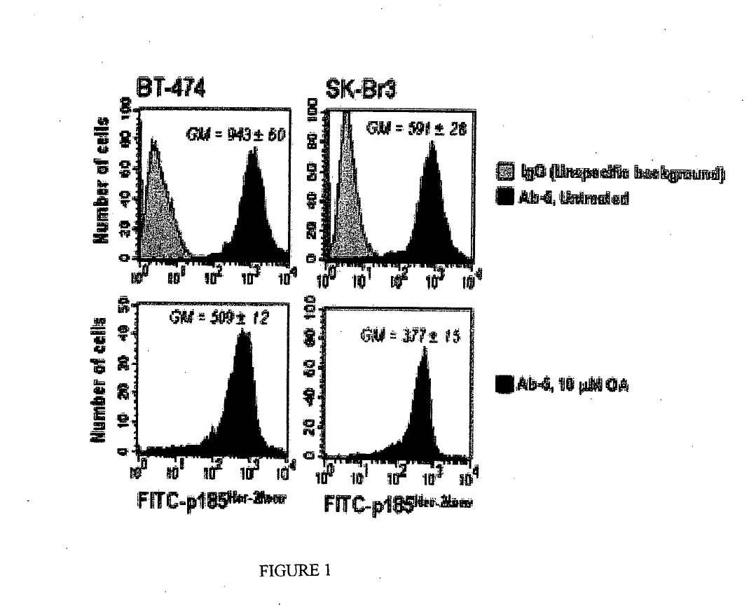

To assess the effects of exogenous supplementation with a fatty acid (OA) on Her-2/neu expression, SK-Br3 and BT-474 cells, after a 24-hour starvation period in medium without serum, were incubated for 48 hours with 10 mM of OA complexed to BSA in low-serum (0.1% FBS) conditions. The cell surface-associated expression of Her-2/neu-encoded p185^{Her-2/neu} oncoprotein was then determined by measuring the binding of a mouse monoclonal antibody directed against the ectodomain of p185^{Her-2/neu} (Ab-5 clone) in OA-treated BT-474 and SK-Br3 cells. Flow cytometric analysis of cell surface-associated p185^{Her-2/neu} demonstrated a significant reduction of p185^{Her-2/neu} expression levels in BT-474 breast cancer cells following OA treatment (up to 46% reduction at 10 mM OA; FIG. 1). Her-2/neu-overexpressing SK-Br3 breast cancer cells were also sensitive to the down-regulatory effects of OA on Her-2/neu expression (up to 36% reduction at 10 mM OA; FIG. 1). These findings reveal that exogenous supplementation significantly down-regulates p185^{Her-2/neu} overexpression in breast cancer cells harboring amplification of the Her-2/neu oncogene. Indeed, this down-regulatory effect was comparable to that found following exposure to optimal concentrations of trastuzumab (up to 48% reduction at 20 mg/ml trastuzumab).

### EXAMPLE 3

### Exogenous Supplementation with a Fatty Acid Synergistically Enhanced Trastuzumab-induced Down-regulation of Her-2/neu

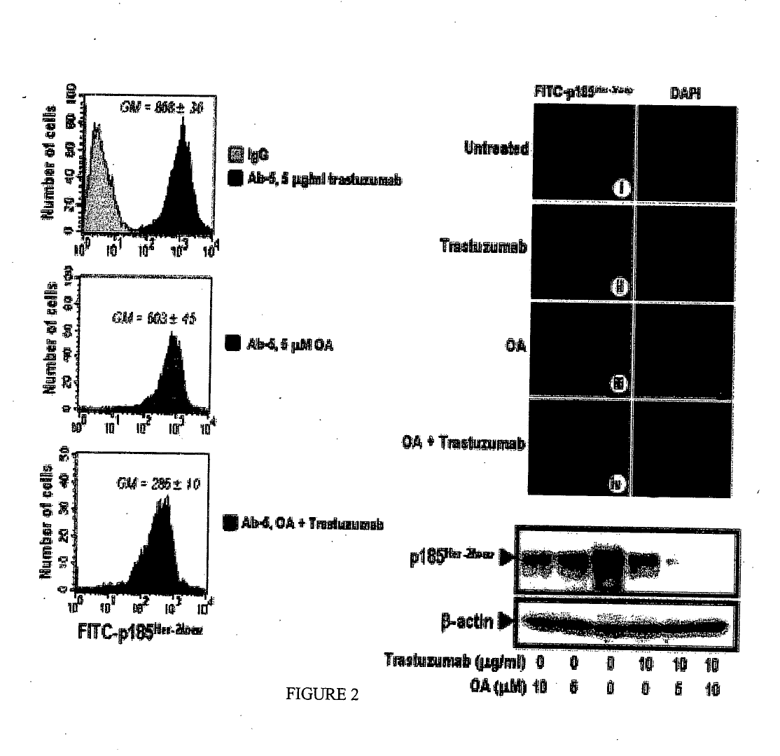

The down-regulatory effects of the fatty acid OA on p185^{Her-2/neu} expression indicate that exogenous supplementation with OA may sensitize breast cancer cells to the well-known p185^{Her-2/neu} down-regulatory actions of trastuzumab [46-48]. To assess this effect, cell surface-associated p185^{Her-2/neu} was first measured by flow cytometry following treatment with low doses of OA (5 mM) and trastuzumab (5 mg/ml). Remarkably, the concurrent combination of OA and trastuzumab reduced p185^{Her-2/neu} expression more than when either agent was administered alone (FIG. 2A). OA and trastuzumab co-treatment induced a 70% decrease of p185^{Her-2/neu} expression when concurrently combined in BT-474 breast cancer cells, whereas when used alone, OA and trastuzumab caused a 36% and an 8% down-regulation of p185^{Her-2/neu}, respectively. In SK-Br3 cells, combined treatment with OA resulted in a synergistic increase in the trastuzumab-mediated down-regulation of p185^{Her-2/neu} up to 65%, whereas a 24% and 26% reduction in p185^{Her-2/neu} expression was observed following treatment with OA or trastuzumab as single agents, respectively.

The impact of OA supplementation in the subcellular localization of p185^{Her-2/neu} in Her-2/neu-overexpressing breast cancer cells was also investigated. To address this question, BT-474 cells, at 48 hours after treatment with OA, trastuzumab, or OA plus trastuzumab, were permeabilized with Triton X-100 for the intracellular delivery of antibodies. Thereafter, p185^{Her-2/neu} cellular localization was assessed using the anti-erbB2, Ab-3 mouse monoclonal antibody (Oncogene Research Products), which is directed against the C-terminal 14 amino acids of p185^{Her-2/neu}. Untreated BT-474 cells showed a prominent cell-surface staining of p185^{Her-2/neu} whereas, upon OA treatment, p185^{Her-2/neu}-associated membrane staining was markedly reduced (FIG. 2B). Indeed, p185^{Her-2/neu} oncoprotein in OA-treated BT-474 cells displayed a cellular distribution similar to that induced by the anti-p185^{Her-2/neu} antibody trastuzumab because it was, to some extent, distributed throughout the cytoplasm. Equivalent results were found in SK-Br3 breast cancer cells. Of note, almost a negative staining of cell surface-associated p185^{Her-2/neu} was observed following the co-exposure of BT-474 to OA and trastuzumab. Western blotting analyses further confirmed that a dramatic down-regulation of p185^{Her-2/neu} takes place in trastuzumab-treated Her-2/neu-overexpressing human breast cancer cells in the presence of increasing concentrations of OA, while the levels of β-actin remained unchanged (FIG. 2C). These findings demonstrated that OA, similarly to trastuzumab, selectively down-regulates expression of the p185^{Her-2/neu} oncoprotein in human breast cancer cells. Moreover, a synergistic augmentation of trastuzumab-induced down-regulation of p185^{Her-2/neu} expression occurred in OA-supplemented breast cancer cells, further indicating that Her-2/neu down-regulation is attributable to OA in human breast cancer cells.

The data described in this Example and in other Examples is entered on the effects of oleic acid, an exemplary fatty acid of the ω-9 monounsaturated class. Other fatty acids conforming to the general definition of useful fatty acids are also known and have been shown to be effective. See Table 2.

TABLE 2
Effect of Fatty Acids on Her-2/neu-overexpressing cells

| Fatty Acid | Lipogenic Cells | Breast Cancer Cells |
|---|---|---|
| ω-9 Oleic acid | No effect | Down-regulates |
| | | FAS at high |
| | | concentrations |
| ω-6 Linoleic acid | Potent | No effect |
| | Down-regulator | |
| ω-6 Arachidonic acid | Potent | No effect |
| | Down-regulator | |
| ω-6 Gamma-Linolenic | No effect | Potent Down-regulator |
| acid | | |
| ω-3 Alpha-Linolenic | No effect | Potent Down-regulator |
| acid | | |
| ω-3 Docosahexaenoic | No effect | Weak Down-regulator |
| acid | | |
| ω-3 Eicosapentaenoic | Down-regulator | No effect |
| acid | | |

### EXAMPLE 4

### Exogenous Supplementation with a Fatty Acid Synergistically Enhanced Trastuzumab-induced Inhibition of Cell Growth in Her-2/neu-overexpressing Breast Cancer Cells

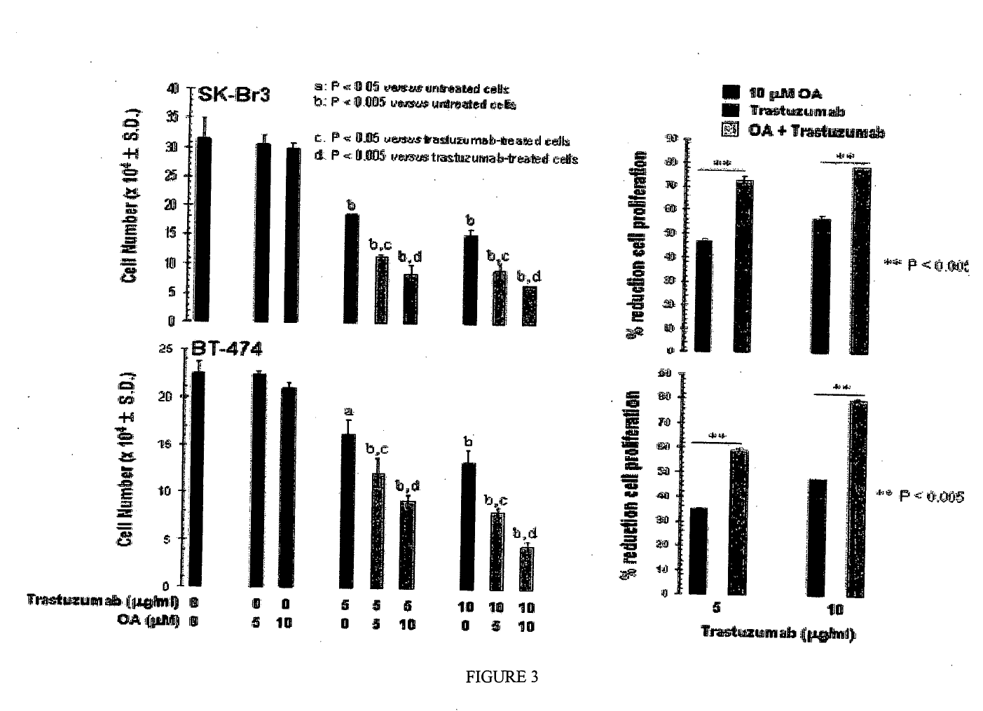

The effects of a concurrent combination of OA and trastuzumab on the anchorage-dependent growth properties of Her-2/neu-overexpressing breast cancer cells were also assessed. As expected, the anchorage-dependent cell growth of Her-2/neu overexpressing was significantly decreased in the presence of increasing concentrations of trastuzumab, while exogenous supplementation with low concentrations of OA had no notable effects on breast cancer cell proliferation. Interestingly, when added in the presence of OA, trastuzumab further inhibited cell proliferation of SK-Br3 and BT-474 cells (FIG. 3A, left panels). Moreover, the increase in growth inhibition with the addition of OA over that of trastuzumab itself was statistically significant (FIG. 3A, right panels), indicating a synergistic interaction between OA and trastuzumab during growth inhibition of Her-2/neu-overexpressing breast cancer cells.

Next, the cytotoxic interactions between OA and trastuzumab and their effects on SK-Br3 and BT-474 cells were examined by evaluating the metabolic status of breast cancer cells co-treated with trastuzumab and OA, as judged by the mitochondrial conversion of the tetrazolium salt, MTT, to its formazan product (MTT assay). First, we measured the changes in cell toxicity of 10 μg/ml trastuzumab after 72 hours of co-exposure to increasing concentrations of OA. The simultaneous presence of OA during the incubation period with trastuzumab caused a significant increase in the cytotoxic effects of trastuzumab (FIG. 3B, left panels). Next, we evaluated the reduction in trastuzumab concentrations needed for a 50% decrease in cell viability (IC₅₀) following OA supplementation. The IC₅₀ values of trastuzumab were measured after 72 hours of treatment in the presence or absence of a given concentration of OA, and the degree of potentiation of trastuzumab efficacy was expressed as a sensitization factor by dividing the IC₅₀ values in the absence of OA by those in the presence of OA. The data showed that OA exposure dramatically enhanced the cytotoxic activity of trastuzumab. The most significant changes were seen in BT-474 cells, in which co-exposure to 10 mM OA decreased the IC₅₀ value of trastuzumab from about 30 mg/ml to 0.75 mg/ml (40-fold sensitization factor). For SK-Br3 cells, OA co-exposure decreased the IC₅₀ value of trastuzumab from about 40 mg/ml to 1.5 mg/ml (27-fold sensitization factor). The precise nature of the interaction between trastuzumab and OA was investigated further using the classical Berenbaum isobologram analysis. When the experimental isoeffect points (the concentrations of trastuzumab and OA that, when combined, produced a 50% reduction in survival of BT-474 and SK-Br3 cells) were plotted and compared with the additive line, the data points fell to the left of the line, indicating a supra-additive or synergistic interaction between the two agents (FIG. 3B, right panels). While these figures provided a graphical representation of trastuzumab-OA interactions, the values of the mean combination index for the 50% cytotoxic level (CI₅₀) were also calculated. When statistical tests were carried out to evaluate whether significant differences in the CI₅₀ means values occurred as compared with a null-hypothesized CI₅₀ of 1 (additivity), and to evaluate formnally whether synergism was evident, concurrent administration of trastuzumab and OA resulted in a significant synergism in BT-474 and SK-Br3 cells (CI₅₀=0.395 and 0.479, respectively; P<0.005). In other words, the combined quantity of the two agents necessary to reduce BT-474 and SK-Br3 cell viability by 50% was only about 0.4 times the quantity required if they demonstrated purely additive behavior (P<0.005 compared with a null-hypothesized interaction index of 1, i.e., additivity).

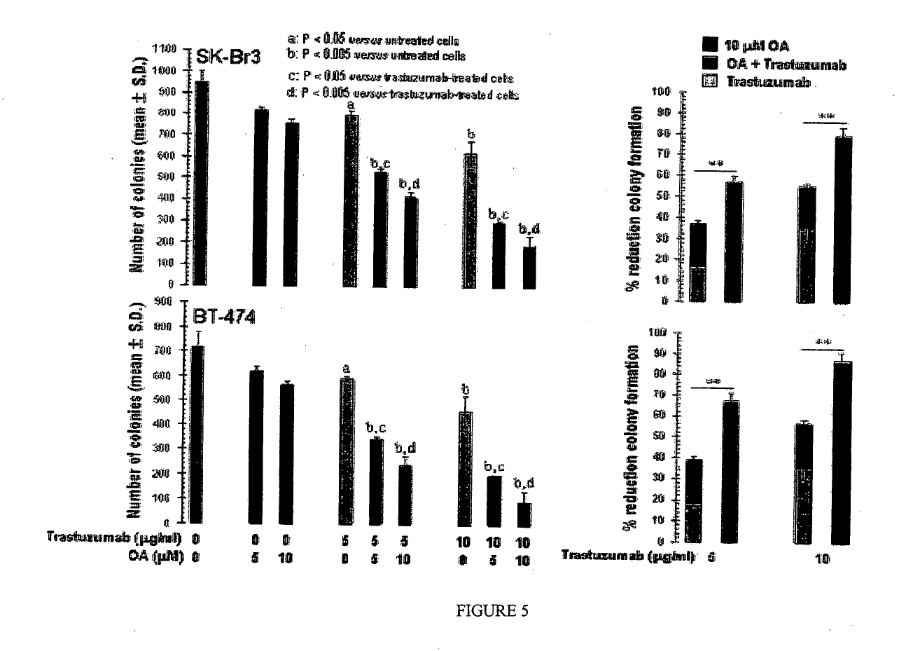

The acquisition of anchorage-independent growth is generally considered to be one of the in vitro properties associated with the malignancy of cells. In fact, colonization of metastatic tumor cells at a distant site may be partially modeled in soft-agar assays. Therefore, the effects of concurrent exposure to trastuzumab and OA on the ability of Her-2/neu overexpressing cells to grow in anchorage-independent conditions were evaluated. As a single agent, OA slightly decreased the ability of SK-Br3 and BT-474 cells to form colonies in soft agar, whereas trastuzumab, as expected, significantly blocked anchorage-independent growth of Her-2/neu overexpressing cells (FIG. 5, left panels). Interestingly, Her-2/neu-dependent, anchorage-independent cell growth was completely abolished in a synergistic manner following co-exposure to OA and trastuzumab (FIG. 5, left and right panels). Taken together, these results demonstrate that exogenous supplementation with OA induces synergistic augmentation of trastuzumab efficacy towards Her-2/neu-overexpressing breast cancer cells.

### EXAMPLE 5

### Exogenous Supplementation with a Fatty Acid Synergistically Enhanced Trastuzumab-induced Apoptotic Cell Death of Her-2/neu-overexpressing Breast Cancer Cells

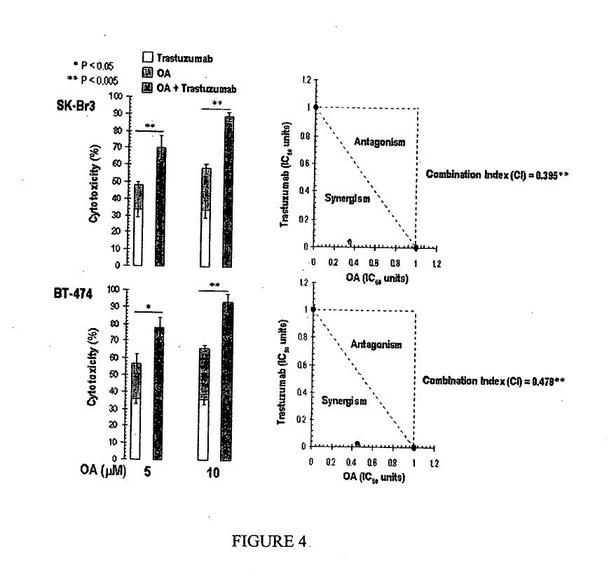

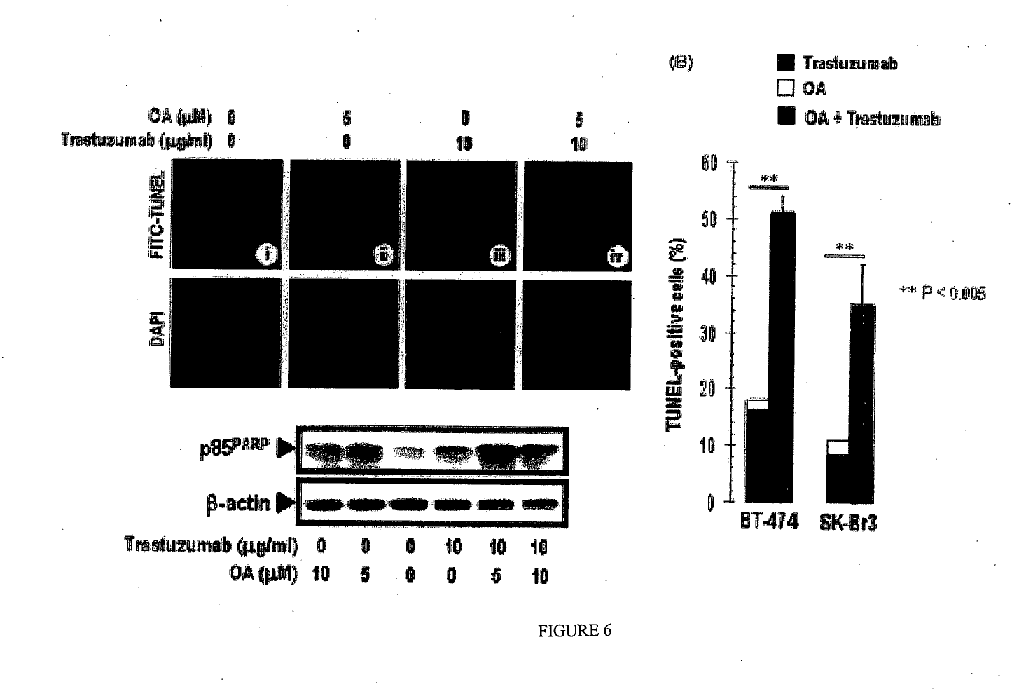

To assess if the synergistic interaction between trastuzumab and OA observed above represented cell death, we next focused on an apoptotic effect of the combination of OA and trastuzumab as measured by the enzymatic in situ labeling of apoptosis-induced DNA double-strand breaks (TUNEL assay). Individually, OA (5 μM) and trastuzumab (10 μg/ml) caused slight increases in the number of apoptotic cells (2% and 16% of TUNEL-positive cells, respectively). Remarkably, there was an impressive increase in apoptosis when BT-474 cells were treated simultaneously with both agents (51% TUNEL-positive cells; FIG. 4A, top panels). The ability of OA to synergistically enhance trastuzumab-induced apoptotic cell death in SK-Br3 cells also demonstrated a synergistic nature. To obtain further evidence that OA synergistically promoted trastuzumab-induced apoptosis, we examined whether the caspase-3-dependent proteolysis of PARP, a hallmark feature of apoptosis, also occurred in Her-2/neu-overexpressing breast cancer cells. Using a rabbit polyclonal antibody specific for the p85 fragment of PARP that results from caspase cleavage of the 116 kDa intact PARP molecule, a small amount of the p85 PARP degradation product was detected in trastuzumab-treated BT-474 cells, whereas an increased cleavage of the death substrate PARP was apparent in BT-474 cells co-treated with trastuzumab and OA (FIG. 6A, bottom panel). Equivalent results were found in SK-Br3 breast cancer cells. These results, taken together, establish that the combined treatment with trastuzumab and OA synergistically enhanced apoptotic cell death of Her-2/neu-overexpressing breast cancer cells.

### EXAMPLE 6

### Exogenous Supplementation with OA Synergistically Enhanced Trastuzumab-induced Up-regulation and Nuclear Accumulation of p27^{Kip1}

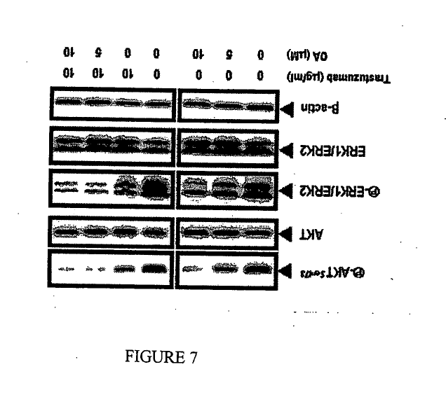

The signaling pathways downstream of Her-2/neu that regulate cell cycle progression and/or cell death were investigated to assess whether they were modified by OA. The treatment of cancer cells with trastuzumab results not only in down-regulation of p185^{Her-2/neu}, but also in further downstream cellular events, including accumulation of the cyclin-dependent kinase inhibitor p27^{Kip1} [42-44, 49]. The cyclin-dependent kinase inhibitor (CDKi) p27^{Kip1} plays a key role in the onset and progression of Her-2/neu-induced breast tumorigenesis and breast cancer progression, and is further involved in the development of trastuzumab resistance [42, 43, 49-51]. A slight increase in the expression of p27^{Kip1} was observed after the treatment of BT-474 cells with suboptimal concentrations of OA. The expression of p27^{Kip1} was significantly enhanced in the presence of trastuzumab. Remarkably, a dramatic up-regulation of p27^{Kip1} expression was observed in trastuzumab-treated BT-474 cells in the presence of increasing concentrations of OA (FIG. 7, left panel).

The effect of an OA-induced interruption of Her-2/neu-dependent signaling on the cellular localization of p27^{Kip1} was also evaluated. Using immunofluorescence microscopy, most of the p27^{Kip1} was found in the cytosol of proliferating BT-474 cells. Treatment with suboptimal concentrations of trastuzumab resulted in a significant translocation of immunufluorescent p27^{Kip1} from cytosol to cell nuclei, while co-treatment with OA resulted in an almost complete translocation of p27^{Kip1} from cytosol to cell nuclei (FIG. 7, right panel). Without wishing to be bound by theory, it is believed that this remarkable up-regulation and nuclear accumulation of p27^{Kip1} plays a pivotal role in determining the enhanced apoptotic cell death of trastuzumab-treated breast cancer cells following OA co-exposure.

### EXAMPLE 7

### Exogenous Supplementation with OA Inhibits Her-2/neu-driven MAPK and AKT Phosphoproteins

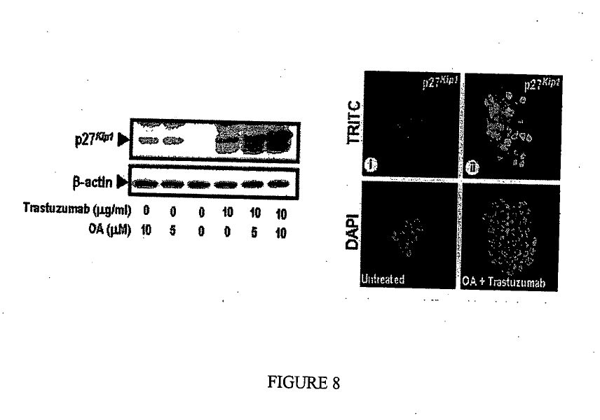

The signaling pathways down-stream of Her-2/neu that regulate cell cycle progression and/or cell death were examined for effects following exogenous supplementation with OA. In BT-474 cells, OA treatment inhibited active MAPK and active AKT as measured by antibodies specific to phospho-MAPK and phospho-Ser⁴⁷³ AKT, respectively, without changes in total MAPK and total AKT (FIG. 8). Consistent with earlier studies [44], trastuzumab treatment in BT-474 cells significantly inhibited MAPK as well as AKT function, as measured by steady-state levels of phosphorylated MAPK and phospho-Ser⁴⁷³ AKT, respectively (FIG. 8). Although the exquisite sensitivity of MAPK and AKT signaling pathways to the down-regulatory effects of either OA or trastuzumab as single agents affected the ability to demonstrate a synergistic blocking effect of active MAPK and AKT following co-exposure to OA and trastuzumab, low doses of OA (5 μM) were found to significantly enhance the ability of trastuzumab (10 μg/ml) to reduce the activation status of these phosphoproteins.

### EXAMPLE 8

### Effect of γ-linolenic Acid on Her-2/neu-overexpressing Cells Involved in a Variety of Cancers

The ω-6 poly-unsaturated fatty acid γ-linolenic acid (GLA; 18:3n-6) was analyzed for a possible effect on the expression of the Her-2/neu (erbB-2) oncogene, which is involved in development of numerous types of human cancer. Flow cytometric and immunoblotting analyses demonstrated that GLA treatment substantially reduced Her-2/neu protein levels in the Her-2/ neu-overexpressing cell lines BT-474, SK-Br3, and MDA-MB-453 (breast cancer), SK-OV3 (ovarian cancer), and NCI-N87 (gastrointestinal tumor derived). GLA exposure led to a dramatic decrease in Her-2/neu promoter activity and a concomitant increase in the levels of polyomavirus enhancer activator 3 (PEA3), a transcriptional repressor of Her-2/neu, in these cell lines. In transient transfection experiments, a Her-2/neu promoter bearing a mutated PEA3 site was not subject to negative regulation by GLA in Her-2/neu-overexpressing cell lines. Concurrent treatments of Her-2/neu-overexpressing cancer cells with GLA and the anti-Her-2/neu antibody trastuzumab led to synergistic increases in apoptosis as well as reduced growth and colony formation.

The oil from seeds of the evening primrose (and that from seeds of borage and black currant) contains γ-linolenic acid (GLA), a member of the ω-6 family of polyunsaturated fatty acids.

Exogenous supplementation of cultured breast cancer cells with GLA significantly diminished proteolytic cleavage of the extracellular domain of the Her-2/neu-coded p185^{Her-2/neu} tyrosine kinase oncoprotein and, consequently, its activation (16) . Considering that activation and overexpression of the Her-2/neu oncogene are crucial for the etiology, progression, and cell sensitivity to various anti-cancer treatments in about 30% of breast carcinomas (17-32), these findings showed a previously unrecognized mechanism by which GLA might regulate breast cancer cell growth, metastasis formation, and response to chemotherapy and endocrine therapy. Two remaining issues are, first, whether GLA-induced deactivation of p185^{Her-2/neu} relates to GLA-induced changes in Her-2/neu gene expression; and, second, whether the ability of GLA to regulate the Her-2/neu oncogene is a common mechanism of GLA's action against other types of cancer.

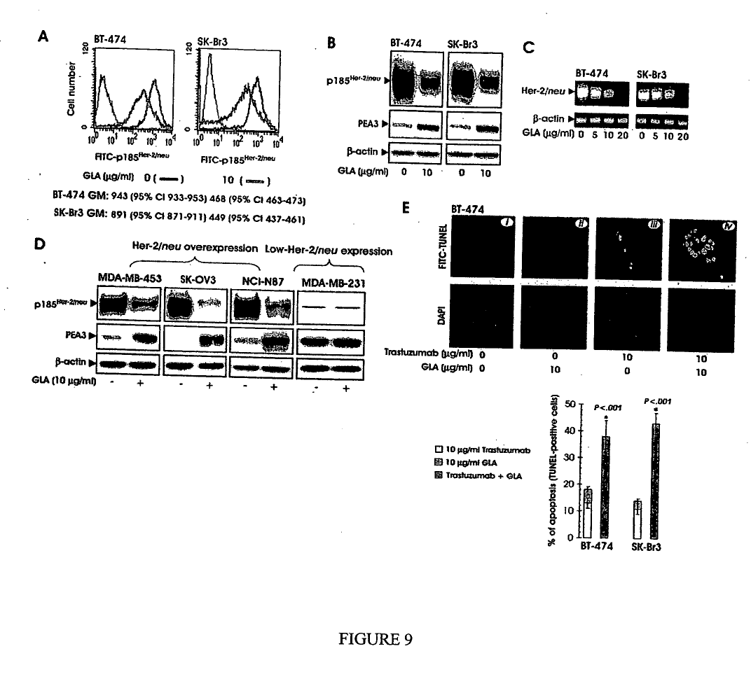

To characterize the effects of GLA on the expression of the Her-2/neu oncogene, BT-474 and SK-Br3 breast cancer cells, which naturally contain amplified copies of the Her-2/neu oncogene (33, 34), were first treated with GLA (10 μg/mL for 48 hours). In flow cytometry analyses, levels of cell surface-associated Her-2/neu protein, i.e., p185^{Her-2/neu} were substantially lower in GLA-treated cells than in vehicle-treated cells (FIG. 9A). Similarly, immunoblot analysis indicated that GLA treatment led to a substantial reduction in Her-2/neu protein levels in both cell lines (FIG. 9B). Although Her-2/neu overexpression was originally attributed solely to erbB-2 gene amplification, an elevation in Her-2/neu mRNA levels per gene copy is also observed in all cell lines that exhibit gene amplification (35).

Reporter gene expression and reverse transcription-polymerase chain reaction (RT-PCR) analyses were undertaken to characterize the effects of GLA on the transcription of the Her-2/neu gene. Treatment of BT-474 and SK-Br3 cells that had been transfected with a construct containing a luciferase reporter gene driven by a wild-type Her-2/neu promoter fragment with GLA (10 μg/mL for 48 hours) led to a strong reduction in reporter gene expression in both lines (Table 3).

For semiquantitative RT-PCR analyses, BT-474 and SK-Br3 cells were treated with varying concentrations of GLA (5, 10, or 20 μg/ml for 48 hours) and then total RNA was extracted from the cells. One microgram of total RNA was then reverse-transcribed and amplified with specific primers for Her-2/neu, and the products were separated on agarose gels (FIG. 9C). Strong, dose-dependent decreases in transcription of the Her-2/neu gene in both cells lines were observed with GLA treatment.

Medication of the GLA-induced repression of Her-2/neu transcription is mediated by the DNA binding protein PEA3 was also investigated. PEA-3, a member of the Ets transcription factor family, specifically targets a DNA sequence in the Her-2/neu promoter (and not in the promoter for genes encoding any other HER isoform), thus suppressing Her-2/neu overexpression in cancer cells (36-38). Immunoblot analysis of BT-474 and SK-Br3 cells that were treated for 48 hours with 10 μg/mL GLA showed an increase in the levels of PEA3 protein in both cell lines relative to levels in control-treated cells (FIG. 9B).

TABLE 3
Luciferase activity in transiently transfected cells

| | Wild-type Her-2/neu promoter | | Mutant Her-2/neu promoter | |
|---|---|---|---|---|
| Cell line | EtOH | GLA | EtOH | GLA |
| BT-474 | 100% | 35% (95% CI = 33-37%) | 15% (95% CI = 11-19%) | 12% (95% CI = 9-15%) |
| SK-Br3 | 100% | 54% (95% CI = 51-57%) | 27% (95% CI = 25-29%) | 21% (95% CI = 18-24%) |
| MDA-MB-453 | 100% | 26% (95% CI = 25-27%) | 10% (95% CI = 8-12%) | 11% (95% CI = 3-19%) |
| SK-OV3 | 100% | 21% (95% CI = 16-26%) | 15% (95% CI = 10-20%) | 14% (95% CI = 6-22%) |
| NCI-N87 | 100% | 38% (95% CI = 34-42%) | 20% (95% CI = 14-26%) | 19% (95% CI = 12-26%) |
| MDA-MB-231 | 100% | 80% (95% CI = 75-85%) | 65% (95% CI = 49% to 81%) | 61% (95% CI = 59-63%) |

The luciferase activities reported in Table 3 were assayed in cells that were transiently transfected with a pGL2-luciferase (Promega Inc., Madison, Wis.) construct containing a luciferase reporter gene under the control of a Her-2/neu promoter fragment containing a wild-type or mutant PEA3 binding site, as previously described by Xing et al. (36), incorporated herein by reference. Cells were transfected using FuGENE 6 transfection reagent (Roche Biochemicals, Indianapolis, Ind.) as directed by the manufacturer and were treated with vehicle (ethanol; EtOH) or γ-linolenic acid (10 μg/mL for 24 hours). Luciferase activity from cell extracts was detected with a Luciferase Assay System (Promega Inc.). Results for all treatments are given as the percentage of luciferase activity relative to that in vehicle-treated cells transfected with the wild-type promoter construct. Data are the means and 95% confidence intervals (CIs) of five experiments, each performed in triplicate.

To examine whether the increased PEA3 levels might mediate the inhibition of Her-2/neu transcription in GLA-treated cells, the effect of GLA on transcription from a Her-2/neu promoter bearing a mutated PEA3 binding sequence, at −33 to −28, that is known to abolish PEA3 binding (36, incorporated herein by reference) was investigated. For this analysis we used the same luciferase reporter gene construct described above but containing the HER-2/neu promoter mutation at the PEA3 binding site (5′-GAG GAA-3′ at −33 to −28 changed to 5′-GAG CTC-3′). The mutant promoter was much less active than the wild-type promoter in untreated control cells (Table 3), consistent with a PEA3 binding site on the Her-2/neu promoter acting as a positive regulatory element necessary for elevated expression of the Her-2/neu oncogene in cancer cells. In addition, transcription from the mutant promoter was not reduced in GLA-treated cells.

To gain insight into the possible role of PEA3 in GLA-mediated repression of Her-2/neu transcription, Her-2/neu and PEA3 protein levels were evaluated by immunoblot analysis in a panel of cancer cells with high or low levels of endogenous Her-2/neu (FIG. 9D). In MDA-MB-453 breast cancer, SK-OV3 ovarian cancer, and NCI-N87 gastrointestinal carcinoma cells, all of which overexpress Her-2/neu, levels of PEA3 protein were low to undetectable in untreated control cells. In cells treated with GLA (10 μg/mL for 48 hours), by contrast, Her-2/neu protein levels were markedly decreased and PEA3 protein expression was increased. In transient transfection experiments with the luciferase reporter gene construct, the activity of the Her-2/neu promoter bearing the intact PEA3 binding site was strongly reduced in all three cell lines after GLA treatment relative to control treatment (Table 3). In addition, mutation of the PEA3 binding site greatly reduced reporter gene activity in untreated cells, and GLA exposure did not further reduce reporter gene activity (Table 3). The effects of GLA were different in MDA-MB-231 breast cancer cells, which naturally express low to undetectable levels of Her-2/neu (FIG. 9D). First, GLA exposure did not affect Her-2/neu protein levels in these cells. Second, MDA-MB-231 cells constitutively exhibited high levels of PEA3 protein, and these levels were not changed substantially following GLA exposure. Finally, the transcriptional activity of the Her-2/neu promoter was reduced only marginally by either GLA treatment or mutation of the PEA3 binding sequence (Table 3). Thus, significant GLA inhibition of p185^{Her-2/neu} expression occurred in cells expressing relatively high levels of that protein, and not in cells expressing normal or subnormal levels of p185^{Her-2/neu}. Relatively high levels of p185^{Her-2/neu} are defined herein as intracellular levels of the protein that detectably and reproducibly exceed the level of p185^{Her-2/neu} found in untreated MDA-MB-231 cells.

The influence of GLA-induced transcriptional repression of Her-2/neu on the growth-inhibitory effects of trastuzumab, a humanized monoclonal antibody that binds with high affinity to Her-2/neu and has therapeutic effects in patients with Her-2/neu-positive breast cancer (39-41), was also investigated. For these analyses, apoptosis in BT-474 cells treated with GLA and/or trastuzumab was determined by terminal deoxynucleotidyltransferase-mediated dUTPbiotin nick end labeling (TUNEL) using the DeadEnd Fluorometric TUNEL System (Promega Inc., Madison, Wis.). Immunofluorescence microscopy revealed many strongly positive nuclei in BT-474 cells treated with both drugs, whereas such nuclei were rare in untreated, GLA-treated, and trastuzumab-treated cells (FIG. 9E, top panel). Counts of apoptotic nuclei from four random fields indicated that 5% (95% confidence interval [CI]=4% to 6%) of the cells underwent apoptosis in the presence of 10 μg/mL GLA, 13% (95% CI=11% to 15%) of cells treated with trastuzumab underwent apoptosis, and 38% (95% CI=32% to 44%) of cells treated with trastuzumab plus GLA underwent apoptosis (FIG. 9E). In SK-Br3 cells, those figures were 3% (95% CI=2 to 4%), 11% (95% CI=9% to 13%), and 43% (95% CI=39% to 47%), respectively. A two-way analysis of variance (ANOVA) showed that concurrent exposure to GLA and trastuzumab synergistically increased the apoptotic effects achieved with GLA and trastuzumab as single agents (FIG. 9E , bottom panel).

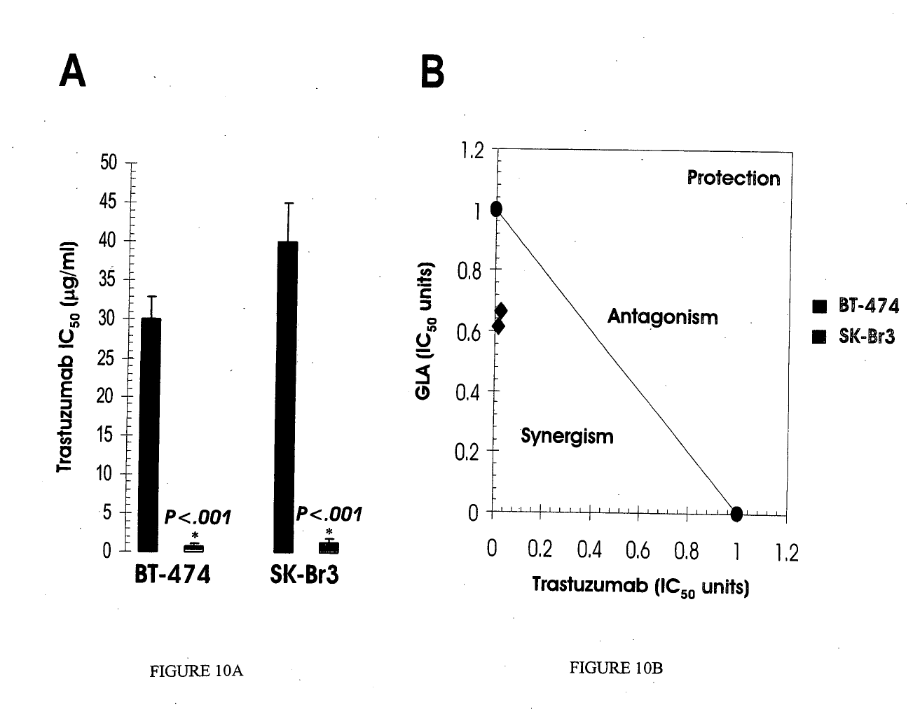

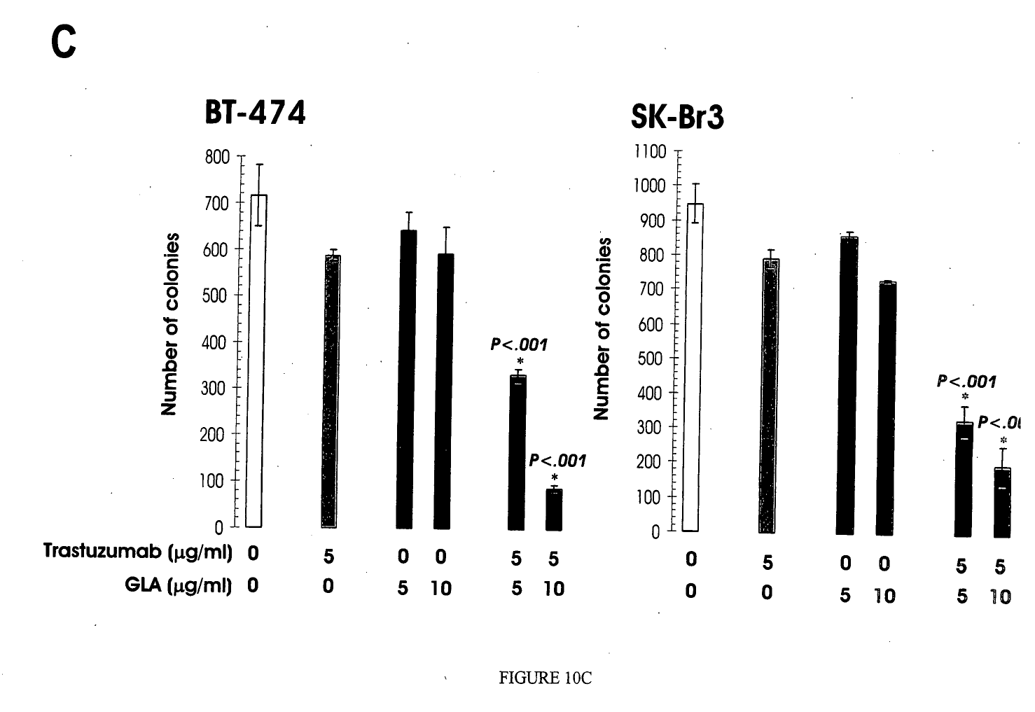

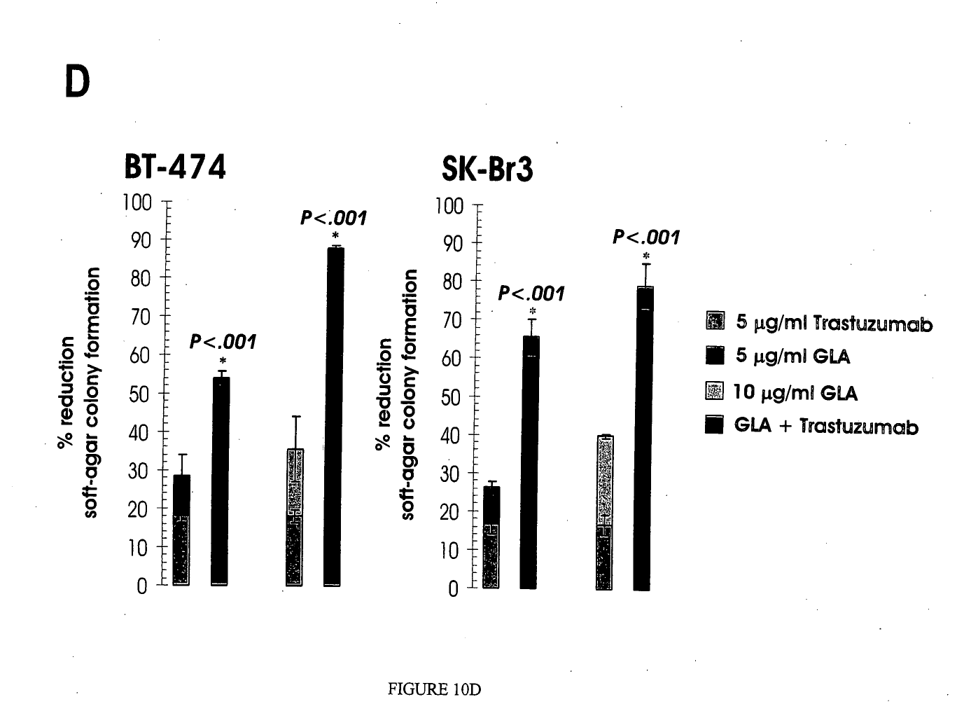

The synergism was also revealed in 3,4,5-dimethylthiazol-2-yl-2,5-diphenyltetrazolium bromide (MTT)—based cell viability assays and by isobologram analysis. Concurrent administration of 10 μg/ml GLA increased the sensitivity of BT-474 and SK-Br3 cells to trastuzumab by approximately 30- and 40-fold, respectively, and two-way ANOVAs showed that these increases were statistically significant. This combination yielded combination index 50 (CI₅₀) values of 0.697 and 0.615 in BT-474 and SK-Br3 cells, respectively, thus suggesting that the interaction was truly synergistic (CI₅₀=1, additive; CI₅₀<1, synergism). When soft-agar assays were used to investigate the actions of GLA on Her-2/neu- induced anchorage-independent cancer cell growth (23, 24), a two-way ANOVA showed that GLA cotreatment enhanced the growth-inhibitory effects of suboptimal doses of trastuzumab in a statistically significant manner in both BT-474 and SK-Br3 breast cancer cell lines (see FIG. 10).

Together, the results indicate that GLA-promoted accumulation of PEA3, a potent repressor of the Her-2/neu promoter (36-38), is a key mechanism underlying GLA-induced suppression of Her-2/neu overexpression in cancer cells. It is possible that GLA activation of other factors that interact with the Her-2/neu promoter, such as AP-2, may account for the reduced Her-2/neu promoter activity in GLA-treated cells; however, AP-2-regulated Her-2/neu promoter regions have different roles in breast and non-breast cancer cells (42-45) and we found that GLA does not appear to regulate AP-2 expression. Considering that GLA mitigates Her-2/neu overexpression by affecting PEA3 binding to the Her-2/ neu promoter, the anti-Her-2/neu actions of GLA should not be affected by the mechanisms of resistance described for trastuzumab (46, 47). Therefore, the disclosed results establish that GLA-induced transcriptional repression of the Her-2/neu oncogene provides a molecular approach to treating Her-2/ neu-overexpressing carcinomas, e.g., in combination with trastuzumab.

### EXAMPLE 9

### Unsaturated Fatty Acids Modulate Her2/neu Gene Expression Via PEA3 in a Variety of Cancer Cells with Elevated FAS

Transient transfection experiments with the human Her-2/neu promoter-driven luciferase gene revealed that OA represses Her-2/neu gene expression in tumor-derived cells exhibiting Her-2/neu gene amplification and overexpression, including SK-Br3 (≦56% reduction), SK-OV3 (≦75% reduction) and NCI-N87 (55% reduction) breast, ovarian and stomach cancer cell lines, respectively. Also, marginal decreases in promoter activity were observed in cancer cells expressing physiological levels of Her-2/neu (<20% reduction in MCF-7 breast cancer cells). Remarkably, OA treatment in Her-2/neu-overexpressing cancer cells was found to induce up-regulation of the Ets protein Polyomavirus Enhancer Activator 3 (PEA3), a transcriptional repressor of Her-2/neu that binds to a PEA3 binding site in the Her2/neu promoter. Also, an intact PEA3 DNA binding site in the endogenous Her-2/neu gene promoter was essential for OA-induced repression of the endogenous gene. Moreover, OA treatment failed to decrease Her-2/neu protein levels in MCF-7/Her2-18 transfectants, which stably express full-length human Her-2/neu cDNA controlled by a SV40 viral promoter. Thus, unsaturated fatty acids such as OA are expected to lower the risk of malignant neoplasms, especially breast and stomach cancer, but also in ovary, colon and endometrium cancer {1-10}.

The preceding examples establish that exogenous supplementation of cultured breast cancer cells with OA significantly down-regulated the expression of Her-2/neu {13}. These findings are significant, in part because no toxicities have been reported or suspected with OA, and supplementation with OA therefore represents a promising dietary intervention for the prevention and/or management of Her-2/neu-related breast, and other, carcinomas {27, 28}. The results provided in this Example show that an unsaturated fatty acid such as OA downregulates Her-2/neu by a mechanism relevant to types of cancer other than breast cancer.

Although overexpression of Her-2/neu both in tumors and in derived cell lines was originally attributed solely to amplification of the erbB-2 gene (usually 2- to 10-fold), an elevation in Her-2/neu mRNA levels per gene copy is also observed in all the cell lines examined exhibiting gene amplification {29}. This indicates that overexpression of the gene precedes and increases the likelihood of gene amplification. Indeed, an increase in transcription rate sufficient to account for the degree of overexpression has been shown in a number of Her-2/neu-overexpressing cancer cell lines {30}. The experiments described in this Example sought to characterize the effects of OA treatment on the transcription rate of Her-2/neu gene. Also addressed is whether the ability of OA to down-regulate Her-2/neu occurs by a common mechanism of OA action towards tumor types reported to exhibit Her-2/neu overexpression, including breast, ovarian and gastric carcinomas. The data show that OA promoted the up-regulation of PEA3, the potent trans-repressor of the human Her-2/neu gene. This up-regulation accounts, at least in part, for the ability of OA to suppress Her-2/neu overexpression in cancer cells. OA-induced transcriptional repression of the Her-2/neu gene is operative in various types of human malignancies, such as breast, ovarian, and stomach carcinomas.

Phenol red-containing Improved Minimal Essential Medium (IMEM) was from Biofluids (Rockville, Md., USA). Oleic acid (18:1n-9) was purchased from Sigma Chemical Co. (St. Louis, Mo., USA). The cultures were supplemented, where indicated, with fatty acid-free bovine serum albumin (FA-free BSA; 0.1 mg/ml) complexed with a specific concentration of OA. A BSA-OA concentrated (100×) solution was formed by mixing 1 ml BSA (10 mg/ml) with various volumes (1-10 μl) of OA (200 mg/ml) in ethanol. The concentrate was mixed for 30 minutes at room temperature before addition to cultures. Control cultures contained uncomplexed BSA.

The primary antibody for Her-2/neu immunoblotting was an anti-p185^{Her-2/neu} mouse monoclonal antibody from Oncogene Research Products (Clone Ab-3; San Diego, Calif., USA). Anti-PEA3 mouse monoclonal (sc-113) and anti-β-actin goat polyclonal antibodies were from Santa Cruz Biotechnology (Santa Cruz, Calif., USA).

SK-Br3 (breast cancer), MDA-MB-231 (breast cancer), SK-OV3 (ovarian cancer), and NCI-N87 (gastrointestinal cancer) cell lines were obtained from the American Type Culture Collection (ATCC). MCF-7 cells stably overexpressing the Her-2/neu oncogene (MCF-7/Her2-18) were generated using conventional techniques. Cells were routinely grown in IMEM containing 5% (v/v) heat-inactivated fetal bovine serum (FBS) and 2 mM L-Glutamine. Cells were maintained at 37° C. in a humidified atmosphere of 95% air/5% CO₂. Cells were screened periodically for Mycoplasma contamination.

To conduct Her-2/neu promoter activity assays, cells were initially transfected using FuGENE 6 transfection reagent (Roche Biochemicals, Indianapolis, Ind.) as directed by the manufacturer. Overnight-serum starved cancer cells seeded into 24-well plates (˜5×10⁴ cells/well) were transfected in low-serum (0.1% FBS) media with 1.5 μg/well of the pGL2-luciferase (Promega, Madison, Wis.) construct containing a luciferase reporter gene driven by either an intact (Her-2/neu wild-type PEA3-binding site-luciferase) or by a mutated (Her-2/neu mutated PEA3-binding site-luciferase) Her-2/neu promoter fragment along with 150 μg/well of the internal control plasmid pRL-CMV, which was used to correct for transfection efficiency. After 18 hours, the transfected cells were washed and incubated with either ethanol (v/v) or 20 μM OA in 0.1% FBS. Approximately 24 hours after treatments, luciferase activities from cell extracts were detected with a luciferase Assay System following manufacturer's instructions (Promega, Madison, Wis., USA) using a Victor²™ Multilabel Counter (Perkin-Elmer Life Sciences).

Following treatments with OA, cells were washed twice with PBS and then lysed in buffer [20 mM Tris (pH 7.5), 150 mM NaCl, 1 mM EDTA, 1 mM EGTA, 1% Triton X-100, 2.5 mM sodium pyrophosphate, 1 mM α-glycerolphosphate, 1 mM Na₃VO₄, 1 μg/ml leupeptin, 1 mM phenylmethylsulfonylfluoride] for 30 minutes on ice. The lysates were cleared by centrifugation in an eppendorf tube (15 minutes at 14,000 rpm at 4° C.). Protein content was determined against a standardized control using the Pierce protein assay kit (Rockford, Ill.). Equal amounts of protein were heated in SDS sample buffer (Laemmli) for 10 minutes at 70° C., subjected to electrophoresis on either 3-8% NuPAGE Tris-Acetate (p185^{Her-2/neu}) or 10% SDS-PAGE (PEA3) and then transferred to nitrocellulose membranes. Non-specific binding on the nitrocellulose filter paper was minimized by blocking for 1 hour at room temperature (RT) with TBS-T [25 mM Tris-HCl (pH 7.5), 150 mM NaCl and 0.05% Tween-20] containing 5% (w/v) non-fat dry milk. The treated filters were washed in TBS-T and then incubated with primary antibodies for 2 hours at room temperature in TBS-T containing 5% (w/v) non-fat dry milk. The membranes were washed in TBS-T, horseradish peroxidase-conjugated secondary antibodies in TBS-T were added for 45 minutes, and immunoreactive bands were detected by enhanced chemiluminescence reagent (Pierce, Rockford, Ill.). Blots were re-probed with an anti-β-actin goat polyclonal antibody (Santa Cruz Biotechnology, Santa Cruz, Calif.) to control for protein loading and transfer. Densitometric values of protein bands were quantified using Scion Imaging Software (Scion Corp., Frederick, Md.).

Data are the mean and 95% confidence intervals (95% CI) of three independent experiments. A two-way ANOVA was used to analyze differences in the percentage of luciferase activity between the treatment and the control groups.

Exogenous supplementation with OA inhibited Her-2/neu gene promoter activity in breast, ovarian and stomach cancer cells.

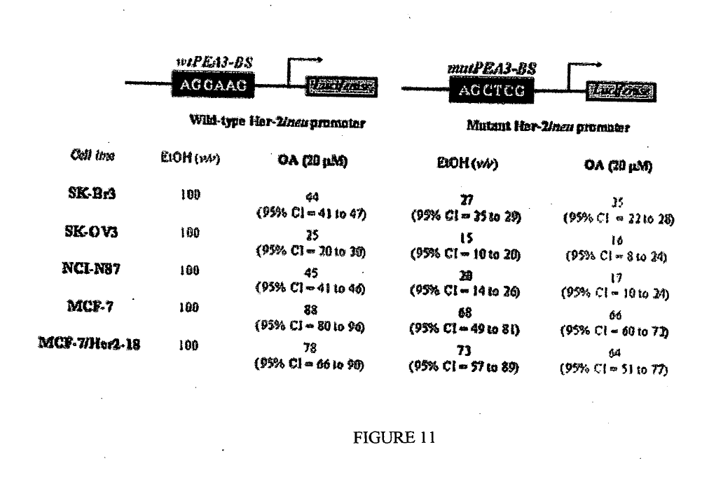

Reporter gene expression was used to characterize the effects of OA on Her-2/neu oncogene transcription. We performed transient transfection experiments with a luciferase reporter gene driven by the wild-type Her-2/neu promoter (pNulit; FIG. 11, top panel). Exogenous supplementation with OA (20 μM; 24 hours) was found to profoundly repress the activity of Her-2/neu gene promoter in SK-Br3 breast cancer cells (up to 56% inhibition; FIG. 11; bottom panel). Accordingly, a significant reduction of the expression levels of Her-2/neu transcripts (Her-2/neu mRNA) was observed in semi-quantitative RT-PCR analyses of RNA isolated from OA-treated SK-Br3 cells. Similar to SK-Br3 breast cancer cells, Her-2/neu-overexpressing SK-OV3 ovarian and NCI-N87 stomach tumor-derived cell lines demonstrated a dramatic reduction (up to 75% and 55% inhibition, respectively) on Her-2/neu gene promoter activity upon OA treatment (FIG. 11; bottom panel).

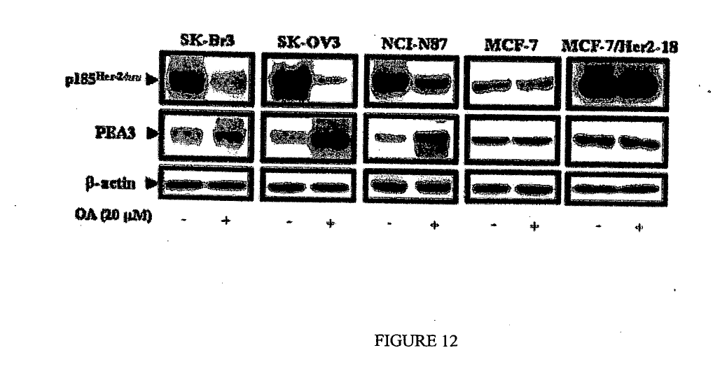

Although the precise molecular mechanisms governing Her-2/neu promoter activity in Her-2/neu-overexpressing cancer cells are far from being totally defined, two main groups of transcription factors, namely the AP-2 and the Ets families of transcription factors, have been shown to be both required for maximal Her-2/neu promoter activity and to be associated with overexpression of the gene in cancer {31}. Recent studies showed that AP-2-regulated Her-2/neu promoter regions have different roles in breast and non-breast cancer cells {31-34}, while the Ets sites appear to be non-tissue specific (“universal”) regulators of Her-2/neu promoter activity {31, 35-36}. In the absence of exogenous supplementation with OA, Her-2/neu-overexpressing SK-Br3, SK-OV3 and NCI-N87 cancer cells did express undetectable to low levels of the DNA-binding protein PEA3, a member of the Ets transcription factor family. PEA3 specifically targets a DNA sequence in the Her-2/neu promoter that suppresses Her-2/neu overexpression and inhibits Her-2/neu-dependent tumorigenesis {35, 36}. Interestingly, a significant up-regulation of PEA3 protein expression, concomitantly with down-regulation of Her-2/neu protein expression, occurred following OA treatment in SK-Br3, SK-OV3 and NCI-N87 cancer cells (FIG. 12). Exogenous supplementation with OA failed to modulate AP-2 expression levels in Her-2/neu-overexpressing cancer cells.

OA-induced transcriptional repression of the Her-2/neu gene requires an intact PEA3 binding site at the endogenous Her-2/neu promoter. To examine whether the increased PEA3 levels might mediate the inhibition of Her-2/neu transcription in OA-treated Her-2/neu-overexpressing cancer cells, the effects of OA on transcription from a promoter bearing a mutated PEA3 binding site (at −33 to −28) that is known to abolish PEA3 binding {35, incorporated herein by reference} were examined. For this analysis the same luciferase reporter gene construct described above, but containing a Her-2/neu promoter mutation at the PEA3 binding site (5′-GAGGAA GAGGAA-3′ (SEQ ID NO: 3) to 5′-GAGCTC-GAGCTC-3′) (FIG. 1, top panel, right; SEQ ID NO: 4), was used. When the levels of wild-type and mutant promoter activities were compared in the absence of OA treatment, the mutant promoter was drastically less active than the wild-type promoter in SK-Br3, SK-OV3 and NCIN86 cells (up to 73, 85 and 80% reductions, respectively). These results establish that a PEA3 binding site in the Her-2/neu promoter acts as a positive regulatory element necessary for elevated expression of the Her-2/neu oncogene in cancer cells {31, 35, 36}. Remarkably, the luciferase reporter gene driven by the promoter containing the mutated PEA3 site was not subject to negative regulation in OA-supplemented SK-Br3, SK-OV3 and NCI-N87 cells (FIG. 11, bottom panel).

The above findings indicated that the formation of inhibitory “PEA3 protein-PEA3 DNA binding site” complexes at the endogenous Her-2/neu promoter could be required for OA-induced transcriptional repression of Her-2/neu gene in Her-2/neu-overexpressing cancer cells. This realization was further supported when the effects of OA treatment on Her-2/neu protein expression, Her-2/neu promoter activity, and PEA3 accumulation were characterized in MCF-7 breast cancer cells, which naturally express physiological (i.e., unelevated) levels of Her-2/neu, and MCF-7 cells engineered to overexpress Her-2/neu under the transcriptional control of a different promoter (i.e., MCF-7/Her2-18 stable transfectants expressing full-length human Her-2/neu cDNA under SV40 promoter control). MCF-7/Her2-18 cells are known to express 45-fold more Her-2/neu than parental MCF-7 cells or the MCF-7/neo control sub-line expressing a neomycin phosphotransferase gene {37}. Her-2/neu and PEA3 protein levels in MCF-7/neo cells were not significantly affected by exogenous supplementation with OA, while the luciferase reporter activity of the wild-type Her-2/neu promoter was slightly reduced by either OA treatment (up to 12% reduction; FIG. 11, bottom panel) or mutation of the PEA3 binding sequence (up to 32% reduction; FIG. 11, bottom panel). Equivalent results were found in wild-type MCF-7 cells. Importantly, there were no important effects of OA supplementation on Her-2/neu gene promoter activity (up to 22% reduction; FIG. 11, bottom panel) and Her-2/neu-PEA3 protein levels in MCF-7/Her2-18 transfectants (FIG. 12).

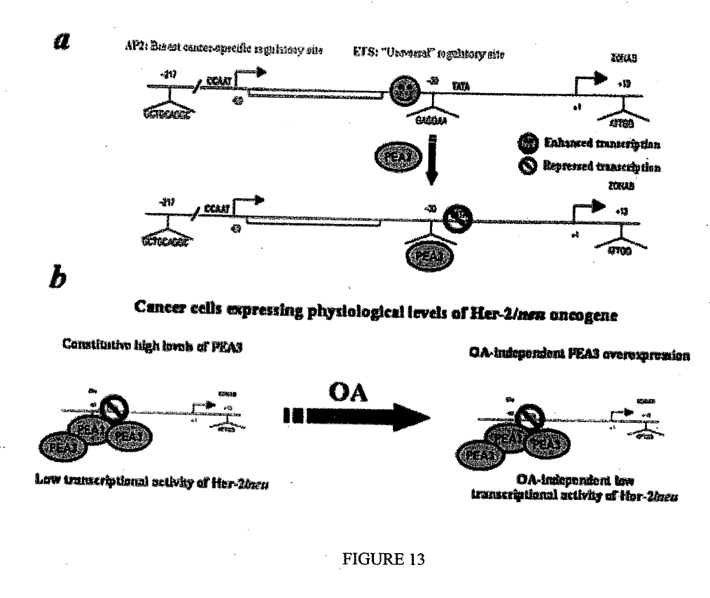

The data provided herein demonstrate that: i) the PEA3 binding motif in the Her-2/neu promoter functions as a positive regulatory element for Her-2/neu gene transcription solely in cancer cells naturally exhibiting both Her-2/neu gene amplification and Her-2/neu protein overexpression (FIG. 13a); ii) there is an inverse correlation between PEA3 and Her-2/neu expression, with low PEA3 expression occurring in Her-2/neu-overexpressing cancer cells and high PEA3 expression occurring in low Her-2/neu-expressing cells, and iii) the ability of OA to down-regulate Her-2/neu promoter activity and to suppress Her-2/neu protein overexpression, while concomitantly up-regulating PEA3 expression, is restricted to cancer cells naturally exhibiting Her-2/neu gene amplification, as OA exposure does not modulate Her-2/neu protein levels when the Her-2/neu gene is overexpressed under the control of a viral promoter. Therefore, PEA3-induced down-regulation of Her-2/neu promoter activity is a major molecular mechanism underlying the anti-Her-2/neu effects observed upon exogenous supplementation with OA of Her-2/neu gene-amplified cancer cells (FIG. 13b,c).

Overexpression of the Her-2/neu oncogene is a frequent molecular event in multiple human cancers. Her-2/neu codes for a transmembrane tyrosine kinase orphan receptor p185^{Her-2/neu} that regulates biological functions as diverse as cellular proliferation, differentiation, motility and apoptosis. Therefore, modulation of Her-2/neu levels must be tightly regulated for normal cellular function. Consistently, in vitro and animal studies demonstrate that deregulated Her-2/neu expression plays a pivotal role in malignant transformation, tumorigenesis and metastasis. Patients with Her-2/neu-overexpressing cancer cells are associated with unfavorable prognosis, shorter relapse time, and low survival rate {14-26}.

The data disclosed herein establish that OA-promoted accumulation of PEA3, the potent trans-repressor of the human Her-2/neu promoter, is a key molecular feature that accounts, at least in part, for the down-regulatory effects of OA on the expression of the Her-2/neu oncogene in cancer cells. The data do not prove, however, that exogenous supplementation with OA exclusively suppresses Her-2/neu overexpression via PEA3. Other Her-2/neu promoter interacting factors, such as AP-2, a member of a family of highly homologous proteins all of which can activate the Her-2/neu promoter {31-34}, may also contribute to the blockade of Her-2/neu promoter activity observed upon OA exposure. While recent studies suggest that AP-2 is not a major player in the increased levels of Her-2/neu transcripts in colon and ovary cancer cells, thus suggesting that the promoter regions leading to Her-2/neu overexpression are different in breast and non-breast cancer cells {34}, no effects of OA on AP-2 levels in Her-2/neu-overexpressing cancer cells were observed. Considering that OA exposure similarly impaired Her-2/neu promoter activity and concomitantly up-regulated PEA3 expression in all the Her-2/neu-overexpressing cell models evaluated, these results support the view that PEA3 and its Ets binding site within the Her-2/neu promoter are the main down-stream effectors involved in OA-induced repression of Her-2/neu oncogene expression in cancer cells, including breast, ovarian and stomach cancer cells (FIG. 13).

The data presented above established that the combined treatment with OA and trastuzumab (Herceptin™), a monoclonal antibody that targets the extracellular domain of Her-2/neu, synergistically increased the extent of apoptotic cell death in Her-2/neu overexpressing cells and strongly impaired the ability of Her-2/neu-overexpressing cancer cells to grow under anchorage-independent conditions. Considering that OA mitigates Her-2/neu overexpression by affecting PEA3 binding to the Her-2/neu promoter, this mechanism of action would not be affected by the mechanisms of resistance described for trastuzumab-based anti-Her-2/neu immunotherapy {46, 47}. The ability of OA to transcriptionally repress Her-2/neu overexpression in a PEA3-dependent manner operates equally in various types of human malignancies, including breast, ovarian and stomach/colon carcinomas.

Each of the following references, cited throughout this disclosure, is incorporated by reference herein in its entirety.

[1.] Welsch C W. Relationship between dietary fat and experimental mammary tumorigenesis: a review and critique. Cancer Res 1992; 52: 2040s-2048s.

[2.] Rose D P. Effects of dietary fatty acids on breast and prostate cancers: evidence from in vitro experiments and animal studies. Am J Clin Nutr 1997; 66: 1513S-1522S.

[3.] Pariza M W. Dietary fat, calorie restriction, ad libitum feeding, and cancer risk. Nutr Rev 1987; 45: 1-7.

[4.] Cohen L A, Rose D P, Wynder E L. A rationale for dietary intervention in postmenopausal breast cancer patients: an update. Nutr Cancer 1993; 19: 1-10.

[5.] Wynder E L, Cohen L A, Muscat J E et al. Breast cancer: weighing the evidence for a promoting role of dietary fat. J Natl Cancer Inst 1997; 89: 766-772.

[6.] Willett W C. Dietary fat and breast cancer. Toxicol Sci 1999; 52: 127-146.

[7.] Lee M M, Lin S S. Dietary fat and breast cancer. Annu Rev Nutr 2000; 20: 221-248.

[8.] Hunter D J, Spiegelman D, Adami H O et al. Cohort studies of fat intake and the risk of breast cancer: a pooled analysis. N Engl J Med 1996; 334: 356-361.

[9.] Holmes M D, Hunter D J, Colditz G A et al. Association of dietary intake of fat and fatty acids with risk of breast cancer. J Am Med Assoc 1999; 281: 914-920.

[10.] Lipworth L, Martinez M E, Angell J et al. Olive oil and human cancer: an assessment of the evidence. Prev Med 1997; 26: 181-190.

[11.] Assman G, De Backer G, Bagnara S et al. International consensus statement of olive oil and the Mediterranean diet: implications for health in Europe. Eur J Cancer Prev 1997; 6: 418-421.

[12.] Martin-Moreno J M, Willett W C, Gorgojo L et al. Dietary fat, olive oil intake and breast cancer risk. Int J Cancer 1994; 58: 774-780.

[13.] Simonsen N R, Fernandez-Crehuet Navajas J, Martin-Moreno J M et al. Tissue stores of individual monounsaturated fatty acids and breast cancer: the EURAMIC study. Am J Clin Nutr 1998; 68:134-141.

[14.] Bartsch H, Nair J, Owen R W. Dietary polyunsaturated fatty acids and cancers of the breast and colorectum: emerging evidence for their role as risk modifiers. Carcinogenesis 1999; 20: 2209-2218.

[15.] Ip C. Review of the effects of trans fatty acids, oleic acid, n-3 polyunsaturated fatty acids, and conjugated linoleic acid on mammary carcinogenesis in animals. Am J Clin Nutr 1997; 66:1523S-1529S.

[16.] Zusman I, Gurevich P, Madar Z et al. Tumor-promoting and tumor-protective effects of high-fat diets on chemically induced mammary cancer in rats. Anticancer Res 1997; 17:349-356.

[17.] Hardy R W, Wickramasinghe N S, Ke S C, Wells A. Fatty acids and breast cancer cell proliferation. Adv Exp Med Biol 1997; 422:57-69.

[18.] Dwyer J T. Human studies on the effects of fatty acids on cancer: summary, gaps, and future research. Am J Clin Nutr 1997; 66:1581S-1586S.

[19.] Martin-Moreno J M. The role of olive oil in lowering cancer risk: in this real gold or simply pinchbeck? J Epidemiol Community Health 2000; 54: 726-727.

[20.] Solanas M, Hurtado A, Costa I et al. Effects of high olive diet on the clinical behavior and histopathological features of rat DMBA-induced mammary tumors compared with a high corn oil diet. Int J Oncol 2002; 21: 745-753.

[21.] Moral R, Solanas M, Garcia G et al. Modulation of EGFR and neu expression by n-6 and n-9 high-fat diets in experimental mammary adenocarcinomas. Oncol Rep 2003; 10: 1417-1424.

[22.] Yamamoto T, Ikawa S, Akiyama T et al. Similarity of protein encoded by the human c-erb-B-2 gene to epidermal growth factor receptor. Nature 1986; 319: 230-234.

[23.] Bargmann C I, Hung M-C, Weinberg R A. The neu oncogene encodes an epidermal growth factor receptor-related protein. Nature 1986; 319: 226-230.

[24.] Slamon D J, Clark G M, Wong S J et al. Human breast cancer: correlation of relapse and survival with amplification of the HER-2/neu oncogene. Science 1987; 235: 177-182.

[25.] Slamon D J, Godolphin W, Jones L A et al. Studies of the HER-2/neu proto-oncogene in human breast and ovarian cancer. Science 1989; 244: 707-712.

[26.] Ross J S, Fletcher J A. The HER-2/neu oncogene in breast cancer: prognostic factor, predictive factor, and target for therapy. Stem Cells 1998; 16: 413-428.

[27.] Di Fiore P P, Pierce J H, Kraus M H et al. erbB-2 is a potent oncogene when overexpressed in NIH/3T3 cells. Science 1987; 237: 178-182.

[28.] Hudziak R M, Schlessinger J, Ullrich A. Increased expression of the putative growth factor receptor p185HER2 causes transformation and tumorigenesis of NIH 3T3 cells. Proc Natl Acad Sci USA 1987; 84:7159-7163.

[29.] DiGiovanna M P, Chu P, Davison T L et al. Active signaling by HER-2/neu in a subpopulation of HER-2/neu-overexpressing ductal carcinoma in situ: clinicopathological correlates. Cancer Res 2002; 62:6667-6673.

[30.] Xu R, Perle M A, Inghirami G et al. Amplification of Her-2/neu in Her-2/neu-overexpressing and nonexpressing breast carcinomas and their synchronous benign, premalignant, and metastatic lesions detected by FISH in archival material. Modem Pathol 2002; 15:116-124.

[31.] Hoque A, Sneige N, Sahin A A et al. HER-2/neu gene amplification in ductal carcinoma in situ of the breast. Cancer Epidemiol Biomark Prev 2002; 11: 587-590.

[32.] Tan M, Yao J, Yu D. Overexpression of the c-erbB-2 gene enhanced intrinsic metastasis potential in human breast cancer cells without increasing their transformation abilities. Cancer Res 1997; 57:1199-1205.

[33.] Eccles S A. The role of c-erbB-2/HER2/neu in breast cancer progression and metastasis. J Mammary Gland Biol Neoplasia 2001; 6:393-406.

[34.] Yu D, Hung M C. Role of erbB2 in breast cancer chemosensitivity. Bioessays 2000; 22: 673-680.

[35.] Dowsett M. Overexpression of HER-2 as a resistance mechanism to hormonal therapy for breast cancer. Endocrinol Relat Cancer 2001; 8:191-195.

[36.] Carter P, Presta L, Gorman C M et al. Humanization of an antip185HER2 antibody for human cancer therapy. Proc Natl Acad Sci US A 1992; 89: 4285-4289.

[37.] Baselga J, Tripathy D, Mendelsohn J et al. Phase II study of weekly intravenous recombinant humanized anti-p185HER2 monoclonal antibody in patients with HER2/neu-overexpressing metastatic breast cancer. J Clin Oncol 1996; 14: 737-744.

[38.] Cobleigh M A, Vogel C L, Tripathy D et al. Multinational study of the efficacy and safety of humanized anti-HER2 monoclonal antibody in women who have HER2-overexpressing metastatic breast cancer that has progressed after chemotherapy for metastatic disease. J Clin Oncol 1999; 17: 2639-2648.

[39.] Vogel C L, Cobleigh M A, Tripathy D et al. Efficacy and safety of trastuzumab as a single agent in first-line treatment of HER2-overexpressing metastatic breast cancer. J Clin Oncol 2002; 20:719-726.

[40.] Slamon D J, Leyland-Jones B, Shak S et al. Use of chemotherapy plus a monoclonal antibody against HER2 for metastatic breast cancer that overexpresses HER2. N Engl J Med 2001; 344:783-792.

[41.] Lu Y, Zi X, Zhao Y et al. Insulin-like growth factor-I receptor signaling and resistance to trastuzumab (Herceptin). J Natl Cancer Inst 2001; 93: 1852-1857.

[42.] Lu Y, Zi X, Pollak M. Molecular mechanisms underlying IGF-Iinduced attenuation of the growth-inhibitory activity of trastuzumab (Herceptin) on SKBR3 breast cancer cells. Int J Cancer 2004; 108:334-341.

[43.] Nahta R, Takahashi T, Ueno N T et al. p27(kip1) down-regulation is associated with trastuzumab resistance in breast cancer cells. Cancer Res 2004; 64: 3981-3986.

[44.] Yakes F M, Chinratanalab W, Ritter C A et al. Herceptin-induced inhibition of phosphatidylinositol-3 kinase and Akt is required for antibody-mediated effects on p27, cyclin DI, and antitumor action. Cancer Res 2002; 62: 4132-4141.

[45.] Berenbaum M C. What is synergy? Pharmacol Rev 1989; 41: 93-141.

[46.] Sliwkowski M X, Lofgren J A, Lewis G D et al. Non-clinical studies addressing the mechanism of action of trastuzumab (Herceptin). Semin Oncol 1999; 26: 60 70.

[47.] De Santes K, Slamon D, Anderson S K et al. Radiolabeled antibody targeting of the HER-2/neu oncoprotein. Cancer Res 1992; 52:1916-1923.

[48.] Klapper L N, Waterman H, Sela H, Yarden Y. Tumor-inhibitory antibodies to HER-2/ErbB-2 may act by recruiting c-Cbl and enhancing ubiquitination of HER-2. Cancer Res 2000; 60:3384-3388.

[49.] Muraoka R S, Lenferink A E, Law B et al. ErbB2/Neu-induced, cyclin DI-dependent transformation is accelerated in p27-haploinsufficient mammary epithelial cells but impaired in p27-null cells. Mol Cell Biol 2002; 22: 2204-2219.

[50.] Hulit J, Lee R J, Russell R G, Pestell R G. ErbB-2-induced mammary tumor growth: the role of cyclin DI and p27Kipl. Biochem Pharmacol 2002; 64: 827-836.

[51.] Kute T, Lack C M, Willingham M et al. Development of Herceptin resistance in breast cancer cells. Cytometry 2004; 57A:86-93.

[52.] Daly R J. Take your partners, please-signal diversification by the erbB family of receptor tyrosine kinases. Growth Factors 1999; 16:255-263.

[53.] Yarden Y, Sliwkowski M X. Untangling the ErbB signalling network. Nat Rev Mol Cell Biol 2001; 2: 127-137.

[54.] Menard S, Pupa S M, Campiglio M, Tagliabue E. Biologic and therapeutic role of HER2 in cancer. Oncogene 2003; 22:6570-6578.

[55.] DiGiovanna M P. Clinical significance of HER-2/neu overexpression. Part I. In De Vita V T Jr, Hellman S, Rosenberg S A (eds): Principles and Practice of Oncology, Vol. 13 No. 9. Cedar Nolls: Lippincott Williams & Wilkins 1999.

[56.] DiGiovanna M P. Clinical significance of HER-2/neu overexpression. Part II. In De Vita V T Jr, Hellman S, Rosenberg S A (eds): Principles and Practice of Oncology, Vol. 13 No. 10. Cedar Nolls: Lippincott Williams & Wilkins 1999.

[57.] Glauert H P. Dietary fat, gene expression and carcinogenesis. In Berdanier C D, Hargrove J L (eds): Nutrition and Gene Expression CRC Press Inc., FL. 1993; 247-268.

[58.] Xing X, Wang S C, Xia W et al. The ets protein PEA3 suppresses HER-2/neu overexpression and inhibits tumorigenesis. Nat Med 2000; 6: 189-195.

[59.] Wang S C, Hung M C. Transcriptional targeting of the HER-2/neu oncogene. Drugs Today 2000; 36: 835-843.

[60.] Wang S C, Hung M C. HER2 overexpression and cancer targeting. Semin Oncol 2001;28: 115-124.

[61.] Menendez J A, Ropero S, Lupu R, Colomer R. Dietary fatty acids regulate the activation status of Her-2/neu (c-erbB-2) oncogene in breast cancer cells. Ann Oncol 2004; 15:1719-1721.

[62.] Hudelist G, Kostler W J, Attems J et al. Her-2/neu-triggered intracellular tyrosine kinase activation: in vivo relevance of ligand-independent activation mechanisms and impact upon the efficacy of trastuzumab-based treatment. Br J Cancer 2003; 89:983-991.

(1) Horrobin D F. Unsaturated lipids and cancer. In: Horrobin D F, editor. New approaches to cancer treatment. Edinburgh (Scotland): Churchill; 1994. p. 1-29.

(2) Das UN. Gamma-linolenic acid, arachidonic acid, and eicosapentaenoic acid as potential anticancer drugs. Nutrition 1990; 6: 429-34.

(3) Jiang W G, Bryce R P, Horrobin D F. Essential fatty acids: molecular and cellular basis of their anti-cancer action and clinical implications. Crit Rev Oncol Hematol 1998; 27: 179-209.

(4) Begin M E, Ells G, Das U N, Horrobin D F. Differential killing of human carcinoma cells supplemented with n-3 and n-6 polyunsaturated fatty acids. J Natl Cancer Inst 1986; 77: 1053-62.

(5) Begin M E, Ells G, Horrobin D F. Polyunsaturated fatty acid-induced cytotoxicity against tumor cells and its relationship to lipid peroxidation. J Natl Cancer Inst 1988; 80:188-94.

(6) Menendez J A, del Mar Barbacid M, Montero S, Sevilla E, Escrich E, Solanas M, et al. Effects of gamma-linolenic acid and oleic acid on paclitaxel cytotoxicity in human breast cancer cells. Eur J Cancer 2001; 37: 402-13.

(7) Menendez J A, Ropero S, del Barbacid M M, Montero S, Solanas M, Escrich E, et al. Synergistic interaction between vinorelbine and gamma-linolenic acid in breast cancer cells. Breast Cancer Res Treat 2002; 72: 203-19.

(8) Menendez J A, Ropero S, Lupu R, Colomer R. Omega-6 polyunsaturated fatty acid gamma-linolenic acid (18:3n-6) enhances docetaxel (Taxotere) cytotoxicity in human breast carcinoma cells. Oncol Rep 2004; 11: 1241-52.

(9) Menendez J A, Colomer R, Lupu R. Omega-6 polyunsaturated fatty acid gamma-linolenic acid (18:3n-6) is a selective estrogen-response modulator in human breast cancer cells: gamma-linolenic acid antagonizes estrogen receptor-dependent transcriptional activity, transcriptionally represses estrogen receptor expression and synergistically enhances tamoxifen and ICI 182,780 (Faslodex) efficacy in human breast cancer cells. Int J Cancer 2004; 109: 949-54.

(10) Kenny F S, Pinder S E, Ellis I O, Gee J M, Nicholson R I, Bryce R P, et al. Gamma linolenic acid with tamoxifen as primary therapy in breast cancer. Int J Cancer 2000 85: 643-48.

(11) van der Merwe C F, Booyens J, Joubert H F, van der Merwe C A. The effect of gamma-linolenic acid, an in vitro cytostatic substance contained in evening primrose oil, on primary liver cancer: a double-blind placebo controlled trial. Prostaglandins Leukot Essent Fatty Acids 1990; 40: 199-202.

(12) Fearon K C, Falconer J S, Ross J A, Carter D C, Hunter J O, Reynolds P D, et al. An open-label phase I/II dose escalation study of the treatment of pancreatic cancer using lithium gammalinolenate. Anticancer Res 1996; 16: 867-74.

(13) Harris N M, Crook T J, Dyer J P, Solomon L Z, Bass P, Cooper A J, Birch B R. Intravesical meglumine gamma-linolenic acid in superficial bladder cancer: an efficacy study. Eur Urol 2002; 42: 39-42.

(14) Harris N M, Anderson W R, Lwaleed B A, Cooper A J, Birch B R, Solomon L Z. Epirubicin and meglumine gamma-linolenic acid: a logical choice of combination therapy for patients with superficial bladder carcinoma. Cancer 2003; 97: 71-8.

(15) Das U N. Occlusion of infusion vessels on gamma-linolenic acid infusion. Prostaglandins Leukot Essent Fatty Acids 2004; 70: 23-32.

(16) Menendez J A, Ropero S, Lupu R, Colomer R. Dietary fatty acids regulate the activation status of Her-2/neu (c-erbB-2) oncogene in breast cancer cells. Ann Oncol 2004; 15: 1719-21.

(17) Slamon D J, Clark G M, Wong S G, Levin W J, Ullrich A, McGuire W L. Human breast cancer: correlation of relapse and survival with amplification of the HER-2/neu oncogene. Science 1987; 235: 177-82.

(18) Slamon D J, Godolphin W, Jones L A, Holt J A, Wong S G, Keith D E, et al. Studies of the HER-2/neu proto-oncogene in human breast and ovarian cancer. Science 1989 244: 707-12.

(19) Yarden Y. Biology of HER2 and its importance in breast cancer. Oncology 2001; 61 Suppl 2: 1-13.

(20) Ross J S, McKenna B J. The HER-2/neu oncogene in tumors of the gastrointestinal tract. Cancer Invest 2001; 19: 554-68.

(21) Xu R, Perle M A, Inghirami G, Chan W, Delgado Y, Feiner H. Amplification of Her-2/neu gene in Her-2/neu-overexpressing and -nonexpressing breast carcinomas and their synchronous benign, premalignant, and metastatic lesions detected by FISH in archival material. Mod Pathol 2002; 15: 116-24.

(22) Hoque A, Sneige N, Sahin A A, Menter D G, Bacus J W, Hortobagyi G N, et al. Her-2/neu gene amplification in ductal carcinoma in situ of the breast. Cancer Epidemiol Biomarkers Prev 2002; 11: 587-90.

(23) Tan M, Yao J, Yu D. Overexpression of the c-erbB-2 gene enhanced intrinsic metastasis potential in human breast cancer cells without increasing their transformation abilities. Cancer Res 1997; 57: 1199-205.

(24) Eccles S A. The role of c-erbB-2/HER2/neu in breast cancer progression and metastasis. J Mammary Gland Biol Neoplasia 2001; 6: 393-406.

(25) Chen X, Yeung TK, Wang Z. Enhanced drug resistance in cells coexpressing ErbB2 with EGF receptor or ErbB3. Biochem Biophys Res Commun 2000; 277: 757-63.

(26) Orr M S, O'Connor P M, Kohn K W. Effects of c-erbB2 overexpression on the drug sensitivities of normal human mammary epithelial cells. J Natl Cancer Inst 2000 92: 987-94.

(27) Nelson N J. Can HER2 status predict response to cancer therapy? J Natl Cancer Inst. 2000; 92: 366-67.

(28) Valagussa P. HER2 status: a statistician's view. Ann Oncol 2001; 12 Suppl 1: S29- S34.

(29) Yamauchi H, Steams V, Hayes D F. The role of c-erbB-2 as a predictive factor in breast cancer. Breast Cancer 2001; 8: 171-83.

(30) Yu D, Hung M C. Role of erbB2 in breast cancer chemosensitivity. Bioessays 2000; 22: 673-80.

(31) Yu D, Hung M C. Overexpression of ErbB2 in cancer and ErbB2-targeting strategies. Oncogene 2000; 19 : 6115-21.

(32) Dowsett M. Overexpression of HER-2 as a resistance mechanism to hormonal therapy for breast cancer. Endocr Relat Cancer 2001; 8: 191-95.

(33) Jarvinen T A, Tanner M, Rantanen V, Barlund M, Borg A, Grenman S, et al. Amplification and deletion of topoisomerase Ilalpha associate with ErbB-2 amplification and affect sensitivity to topoisomerase II inhibitor doxorubicin in breast cancer. Am J Pathol 2000; 156: 839-47.

(34) Yakes F M, Chinratanalab W, Ritter C A, King W, Seelig S, Arteaga C L. Herceptin-induced inhibition of phosphatidylinositol-3 kinase and Akt Is required for antibody-mediated effects on p27, cyclin DI, and antitumor action. Cancer Res 2002; 62: 4132-41.

(35) Kraus M H, Popescu N C, Amsbaugh S C, King C R. Overexpression of the EGF receptor-related proto-oncogene erbB-2 in human mammary tumor cell lines by different molecular mechanisms. EMBO J 1987; 6: 605-10.

(36) Xing X, Wang S C, Xia W, Zou Y, Shao R, Kwong K Y, et al. The ets protein PEA3 suppresses HER-2/neu overexpression and inhibits tumorigenesis. Nat Med 2000; 6: 189-95.

(37) Wang S C, Hung M C. Transcriptional targeting of the HER-2/neu oncogene. Drugs Today (Barc). 2000; 36: 835-43.

(38) Menendez J A, Vellon L, Mehmi I, Oza B P, Ropero S, Colomer R, et al. Inhibition of fatty acid synthase (FAS) suppresses HER2/neu (erbB-2) oncogene overexpression in cancer cells. Proc Natl Acad Sci USA 2004; 101: 10715-20.

(39) Carter P, Presta L, Gorman C M, Ridgway J B, Henner D, Wong W L, et al. Humanization of an anti-p185HER2 antibody for human cancer therapy. Proc Natl Acad Sci USA 1992; 89: 4285-9.

(40) Vogel C L, Cobleigh M A, Tripathy D, Gutheil J C, Harris L N, Fehrenbacher L, et al. Efficacy and safety of trastuzumab as a single agent in first-line treatment of HER2-overexpressing metastatic breast cancer. J Clin Oncol 2002; 20: 719-26.

(41) Slamon D J, Leyland-Jones B, Shak S, Fuchs H, Paton V, Bajamonde A, et al. Use of chemo therapy plus a monoclonal antibody against HER2 for metastatic breast cancer that overexpresses HER2. N Engl J Med 2001; 344: 783-92.

(42) Bosher J M, Williams T, Hurst H C. The developmentally regulated transcription factor AP-2 is involved in c-erbB-2 overexpression in human mammary carcinoma. Proc Natl Acad Sci USA 1995; 92: 744-7.

(43) Bosher J M, Totty N F, Hsuan J J, Williams T, Hurst H C. A family of AP-2 proteins regulates c-erbB-2 expression in mammary carcinoma. Oncogene 1996; 13: 1701-7.

(44) Hurst H C. Update on HER-2 as a target for cancer therapy: the ERBB2 promoter and its exploitation for cancer treatment. Breast Cancer Res 2001; 3: 395-8.

(45) Vemimmen D, Gueders M, Pisvin S, Delvenne P, Winkler R. Different mechanisms are implicated in ERBB2 gene overexpression in breast and in other cancers. Br J Cancer 2003; 89: 899-906.

(46) Lu Y, Zi X, Zhao Y, Mascarenhas D, Pollak M. Insulin-like growth factor-I receptor signaling and resistance to trastuzumab (Herceptin). J Natl Cancer Inst 2001; 93: 1852-7.

(47) Nagata Y, Lan K H, Zhou X, Tan M, Esteva F J, Sahin A A, et al. PTEN activation con tributes to tumor inhibition by trastuzumab, and loss of PTEN predicts trastuzumab resistance in patients. Cancer Cell 2004; 6: 117-27.

{1.} Martin-Moreno J M, Willett P, Gorgojo L, et al. Dietary fat, olive oil intake and breast cancer risk. Int J Cancer 1994; 58: 774-780.

{2.} Landa M C, Frago N, Tres A. Diet and risk of breast cancer in Spain. Eur J Cancer Prev 1994; 3: 313-320.

{3.} Trichopoulou A, Katsouyanni K, Stuver S, et al. Consumption of olive oil and specific food groups in relation to breast cancer risk in Greece. J Natl Cancer Inst 1995; 87: 110-116.

{4.} La Vecchia C, Negri E., Franceschi S., et al. Olive oil, other dietary fats, and the risk of breast cancer. Cancer Causes Control 1995; 6: 545-550.

{5.} Simonsen N R, Femandez-Crehuet N J, Martin-Moreno J M., et al. Tissue stores of individual monounsaturated fatty acids and breast cancer: the EURAMIC study. European Community Multicenter Study on Antioxidants, Myocardial Infarction, and Breast Cancer. Am J Clin Nutr 1998; 68: 134-141.

{6.} Stoneham M, Goldacre M, Seagroatt V, et al. Olive oil, diet and colorectal cancer: an ecological study and a hypothesis. J. Epidemiol. Community Health 2000; 54: 756-760.

{7.} Braga C, La Vecchia C, Franceschi S, et al. Olive oil, other seasoning fats, and the risk of colorectal carcinoma. Cancer 1998; 82: 448-453.

{8.} Tzonou A, Hsieh C, Polychronopoulou A, et al. Diet and ovarian cancer: a case-control study in Greece. Int J Cancer 1993; 55: 411-414.

{9.} Tzonou A, Lipworth L, Kalandidi A, et al. Dietary factors and the risk of endometrial cancer: a case-control study in Greece. Br J Cancer 1996; 73: 1284-1290.

{10.} Zamora-Ardoy M A, Banez Sanchez F, Banez Sanchez C, Alaminos Garcia P. Olive oil: influence and benefits on some pathologies. An Med Intema 2004; 21: 138-142.

{11.} Martin-Moreno J M. The role of olive oil in lowering cancer risk: is the real gold or simply pinchbeck? J. Epidemiol Community Health 2000; 54: 726-727.

{12.} Menendez J A, Ropero S, Lupu R, Colomer R. Dietary fatty acids regulate the activation status of Her-2/neu (c-erbB-2) oncogene in breast cancer cells. Ann Oncol 2004; 15: 1719-1721.

{13.} Menendez J A, Vellon L, Colomer R, Lupu R. Oleic acid, the main monounsaturated fatty acid of olive oil, suppresses Her-2/neu (erbB-2) expression and synergistically enhances the growth inhibitory effects of trastuzumab (Herceptin) in breast cancer cells with Her-2/neu gene amplification. Ann Oncol 2005; 16: 359-371.

{14.} Yamamoto T, Ikawa S, Akiyama T, et al. Similarity of protein encoded by the human c-erb-B-2 gene to epidermal growth factor receptor. Nature 1986; 319: 230-234.

{15.} Bargmann C I, Hung M-C, Weinberg R A. The neu oncogene encodes an epidermal growth factor receptor-related protein. Nature 1986; 319: 226-230.

{16.} Daly R J. Take your partners, please-signal diversification by the erbb family of receptor tyrosine kinases. Growth Factors 1999; 16: 255-263.

{17.} Yarden Y, Sliwkowski M X. Untangling the ErbB signalling network. Nat Rev Mol Cell Biol 2001; 2: 127-137.

{18.} Slamon D J, Clark G M, Wong S G, et al. Human breast cancer: correlation of relapse and survival with amplification of the HER-2/neu oncogene. Science 1987; 235:177-182.

{19.} Slamon D J, Godolphin W, Jones L A, et al. Studies of the HER-2/neu proto-oncogene in human breast and ovarian cancer. Science 1989; 244: 707-712.

{20.} Ross J S, Fletcher J A. The HER-2/neu oncogene in breast cancer: prognostic factor, predictive factor, and target for therapy. Stem Cells 1998; 16: 413-428.

{21.} Di Fiore P P, Pierce J H, Kraus M H, et al. erbB-2 is a potent oncogene when overexpressed in NIH/3T3 cells. Science 1987; 237: 178-182.

{22.} Hudziak R M, Schlessinger J, Ullrich A. Increased expression of the putative growth factor receptor p185HER2 causes transformation and tumourigenesis of NIH 3T3 cells. Proc Natl Acad Sci USA 1987; 84: 7159-7163.

{23.} Tan M, Yao J, Yu D. Overexpression of the c-erbB-2 gene enhanced intrinsic metastasis potential in human breast cancer cells without increasing their transformation abilities. Cancer Res 1997; 57: 1199-1205.

{24.} Eccles S A. The role of c-erbB-2/HER2/neu in breast cancer progression and metastasis. J. Mammary Gland. Biol. Neoplasia 2001; 6: 393-406.

{25.} Yu D, Hung M-C. Role of erbB2 in breast cancer chemosensitivity. Bioessays 2000; 22: 673-680.

{26.} Dowsett M. Overexpression of HER-2 as a resistance mechanism to hormonal therapy for breast cancer. Endocrinol Relat Cancer 2001; 8: 191-195.

{27.} Marchetti P. Natural medicine: a ‘new frontier’ in oncology? Ann Oncol 2005; 16:339-340.

{28.} Nelson R. Oleic acid suppresses overexpression of ERBB2 oncogene. Lancet Oncol 2005; 6:69.

{29.} Kraus M H, Popescu N C, Amrbaugh S C, King C R. Overexpression of the EGF receptor related proto-oncogene erbB-2 in human mammary tumour cell lines by different molecular mechanisms. EMBO J 1987; 6:605-610.

{30.} Bates N P, Hurst H C. Transcriptional regulation of type I receptor tyrosine kinases in the mammary gland. J Mammary Gland Biol Neoplasia 1997; 2: 153-163.

{31.} Hurst H C. Update on HER-2 as a target for cancer therapy. The ERBB2 promoter and its exploitation for cancer treatment. Breast Cancer Res 2001; 3: 395-398.

{32.} Bosher J M, Williams T, Hurst H C. The developmentally regulated transcription factor AP-2 is involved in c-erbB-2 overexpression in human mammary carcinoma. Proc Natl Acad Sci USA 1995; 92: 744-747.

{33.} Bosher J M, Totty N F, Hsuan J J, Williams T, Hurst H C. A family of AP-2 proteins regulates c-erbB-2 expression in mammary carcinoma. Oncogene 1996; 13: 1701-1707.

{34.} Vernimmen D, Gueders M, Pisvin S, Delvenne P, Winkler R. Different mechanisms are implicated in ERBB2 gene overexpression in breast and in other cancers. Br J Cancer 2003; 89: 899-906.

{35.} Xing X, Wang S C, Xia W, et al. The ets protein PEA3 suppresses HER-2/neu overexpression and inhibits tumourigenesis. Nat Med 2000; 6: 189-195.

{36.} Wang S C, Hung M-C. Transcriptional targeting of the HER-2/neu oncogene. Drugs Today (Barc). 2000; 36: 835-843.

{37.} Benz C C, Scott G K, Sarup J C, et al. Estrogen-dependent, tamoxifen-resistant tumourigenic growth of M C F-7 cells transfected with Her-2/neu. Breast Cancer Res Treat 1993; 24: 85-95.

{38.} Carter P, Presta L, Gorman C M, et al. Humanization of an anti-p185HER2 antibody for human cancer therapy. Proc Natl Acad Sci USA 1992; 89: 4285-4289.

{39.} Baselga J, Tripathy D, Mendelsohn J, et al. Phase II study of weekly intravenous recombinant humanized anti-p185HER2 monoclonal antibody in patients with HER2/neu overexpressing metastatic breast cancer. J Clin Oncol 1996; 14: 737-744.

{40.} Cobleigh M A, Vogel C L, Tripathy D. et al. Multinational study of the efficacy and safety of humanized anti-HER2 monoclonal antibody in women who have HER2-overexpressing metastatic breast cancer that has progressed after chemotherapy for metastatic disease. J Clin Oncol 1999; 17: 2639-2648.

{41.} Vogel C L, Cobleigh M A, Tripathy D, et al. Efficacy and safety of trastuzumab as a single agent in first-line treatment of HER2-overexpressing metastatic breast cancer. J Clin Oncol 2002; 20: 719-726.

{42.} Slamon D J, Leyland-Jones B, Shak S, et al. Use of chemotherapy plus a monoclonal antibody against HER2 for metastatic breast cancer that overexpresses HER2. N Engl J Med 2001; 344: 783-792.

{43.} Allen L F, Eiseman I A, Fry D W, Lenehan P F. CI-1033, an irreversible pan-erbB receptor inhibitor and its potential for the treatment of breast cancer. Semin Oncol 2003; 30 (5 Suppl 16): 65-78.

{44.} Burris H A 3rd. Dual kinase inhibition in the treatment of breast cancer: initial experience with the EGFR/ErbB-2 inhibitor lapatinib. Oncologist 2004; 9 Suppl 3: 10-15.

{45.} Wang S C, Zhang L, Hortobagyi G N, Hung M-C. Targeting HER2: recent developments and future directions for breast cancer patients. Semin Oncol 2001; 28 (6 Suppl 18): 21-29.

{46.} Lu Y, Zi X, Zhao Y, Mascarenhas D, Pollak M. Insulin-like growth factor-I receptor signaling and resistance to trastuzumab (Herceptin). J Natl Cancer Inst 2001; 93:1852-1857.

{47.} Nagata Y, Lan K H, Zhou X, et al. PTEN activation contributes to tumour inhibition by trastuzumab, and loss of PTEN predicts trastuzumab resistance in patients. Cancer Cell. 2004; 6:117-127.

{48.} Scott G K, Chang C H, Emy K M, et al Ets regulation of the erbB2 promoter. Oncogene 2000; 19: 2: 153-163.

What is claimed:

1. A method of treating a cancer cell overexpressing p185^{Her-2/neu} and fatty acid synthase comprising administering a therapeutically effective amount of an unsaturated trans-fatty acid to an organism comprising the cancer cell.

2. The method according to claim 1 wherein a nucleic acid expressing p185^{Her-2/neu} comprises a promoter comprising a PEA3 binding site.

3. The method according to claim 1 wherein the organism in need is a human.

4. The method according to claim 1 wherein the cancer is selected from the group consisting of breast cancer, ovarian cancer, prostate cancer, colorectal cancer, bladder cancer, stomach cancer, lung cancer, oral cancer of the tongue and cancer of the endometrium.

5. The method according to claim 1 wherein the fatty acid is selected from the group consisting of C16-C22 fatty acids.

6. The method according to claim 5 wherein the fatty acid is selected from the group consisting of an omega-9 unsaturated fatty acid, an omega-6 unsaturated fatty acid and an omega-3 unsaturated fatty acid.

7. The method according to claim 6 wherein the fatty acid is selected from the group consisting of oleic acid, γ-linolenic acid and α-linolenic acid.

8. A method of treating a cancer cell overexpressing p185^{Her-2/neu} and fatty acid synthase comprising
(a) administering a first anti-cancer therapeutic to an organism comprising the cancer cell, wherein the first anti-cancer therapeutic reduces the activity of p185^{Her-2/neu}, and
(b) delivering a fatty acid according to claim 1 to the organism.

9. The method according to claim 6 wherein the anti-cancer effect of the combined treatment is greater than the additive effect of two separate treatments.

10. The method according to claim 8 wherein the organism is a human.

11. The method according to claim 8 wherein a nucleic acid expresses p185^{Her-2/neu} said nucleic acid comprising a promoter comprising a PEA3 binding site.

12. The method according to claim 8 wherein the cancer is selected from the group consisting of breast cancer, ovarian cancer, prostate cancer, colorectal cancer, bladder cancer, stomach cancer, lung cancer, oral cancer of the tongue and cancer of the endometrium.

13. The method according to claim 8 wherein the fatty acid is selected from the group consisting of oleic acid, γ-linolenic acid and α-linolenic acid.

14. The method according to claim 8 wherein the first anti-cancer therapeutic is an antibody that specifically binds p185^{Her-2/neu}.

15. The method according to claim 14 wherein the antibody is trastuzumab.

16. A method of reducing the risk of developing a cancer comprising a cell over-expressing p185Her-21neu and fatty acid synthase, the method comprising delivering a prophylactically effective amount of a fatty acid according to claim 1.

17. The method according to claim 15 wherein the fatty acid is selected from the group consisting of oleic acid, γ-linolenic acid and α-linolenic acid.

18. A kit for treatment of a cancer cell overexpressing p185^{Her-2/neu} and fatty acid synthase comprising a compound that specifically inhibits the binding activity of p185^{Her-2/neu}, a fatty acid and a protocol for said treatment.

19. The kit according to claim 18 wherein said compound is trastuzumab.

20. The kit according to claim 18 wherein the fatty acid is selected from the group consisting of oleic acid, γ-linolenic acid and α-linolenic acid.
